(19) United States
(12) Patent Application Publication
(10) Pub. No.: US 2015/0045387 A1
(43) Pub. Date: Feb. 12, 2015

(54) Bi-Aromatic And Tri-Aromatic Compounds As NADPH Oxidase 2 (Nox2) Inhibitors

(71) Applicant: The University of Surrey, Surrey (GB)

(72) Inventors: Jian-Mei Li, Surrey (GB); Brendan Howlin, Surrey (GB); Daniel Nathan Meijles, Guildford (GB)

(21) Appl. No.: 14/345,084

(22) PCT Filed: Sep. 17, 2012
(86) PCT No.: PCT/GB2012/000725

§ 371 Date: Mar. 14, 2014

(30) Foreign Application Priority Data
Sep. 16, 2011 (GB) 1116017.3

(51) Int. Cl. C07D 263/32; C07D 217/26; C07C 235/48; C07C 69/94; C07C 49/83

(52) U.S. Cl. CPC C07D 263/32; C07C 69/94; C07C 49/83; C07C 235/48; C07D 217/26
USPC 514/307; 560/61; 514/544; 548/235; 514/374; 568/331; 514/685; 564/179; 514/622; 560/57; 560/59; 546/147

(57) ABSTRACT

Bi- and tri-aromatic compounds of the formula (I) wherein R1 to RIO and X are as defined, are Nox2 inhibitors that are useful as medicaments for the treatment of a disease or condition selected from: cardiovascular diseases, respiratory diseases, inflammatory diseases, cancers, ageing and age related disorders, kidney diseases, neurodegenerative diseases, diabetes and conditions associated with diabetes. The compounds, their preparation and pharmaceutical compositions comprising them are disclosed.

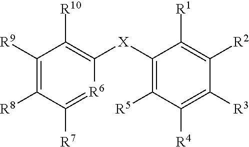

### TECHNICAL FIELD

The present invention relates to novel bi- and tri-aromatic compounds, their preparation, pharmaceutical compositions comprising them, and their use as medicaments for the treatment of diseases or conditions due to their inhibiting effect on nicotinamide adenine dinucleotide phosphate oxidase 2 (NADPH oxidase 2).

### BACKGROUND OF THE INVENTION

Cells generate reactive oxygen species (ROS), such as H₂O₂ and O_{2.}⁻, as by-products of normal cellular metabolism. Nicotinamide adenine dinucleotide phosphate oxidase (NADPH oxidase or NOX) is a multi-component enzyme expressed in almost every cell type in our body and is a major source of ROS generation. Excessive ROS have been identified as major contributors to damage in biological organisms, so-called “oxidative stress”, and are recognized as a key component for the development of many diseases, such hypertension, atherosclerosis, obesity, insulin resistance, diabetes, respiratory disorders, liver diseases, inflammation, and conditions such as ageing.

The Nox family contains at least 7 isoforms (Nox1 to 5 and duox1 and duox2). Among these Noxes, the Nox2 enzyme, also called gp91^{phox}, is different from other members of the Nox family in that it is a highly glycosylated protein, and requires the presence of regulatory subunits, i.e., p40^{phox}, p47^{phox} p67^{phox} and rac1, for generating O_{2.}⁻. It has been reported that in non-phargocytic cells, a substantial proportion of the Nox2 enzyme complex is preassembled under basal physiological conditions, which generates low amounts of O_{2.}⁻ involved mainly in cellular redox-signalling. However, the activity and expression of the Nox2 enzyme can be up-regulated under pathophysiological conditions, and excessive production of O_{2.}⁻ by Nox2 outstrips endogenous antioxidant defence and causes oxidative damage to cells and tissues. O_{2.}⁻ can also serve as a precursor for the generation of other ROS, i.e., hydrogen peroxide (H₂O₂) and peroxynitrite (ONOO.⁻), which may cause further damage to tissues. There is a close relationship between the levels of Nox2 activation and the levels of oxidative damage in many pathological conditions, and, in fact, the activation of Nox2 enzyme can be found in the early course of the development of many diseases (1), (2).

Evidence from human studies points to a link between Nox2 activation and oxidative damage in a variety of diseases and medical conditions. Listed below are examples of diseases and medical conditions in which Nox2-derived oxidative stress has been found to play a key role, in the pathogenesis of the disease or condition, and where an effective therapy to inhibit Nox2 activation and reduce ROS production is urgently needed.

Cardiovascular diseases, such as atherosclerosis, hypertension and heart failure, are major causes of death in the world and are pre-disposed by endothelial dysfunction characterized by excessive ROS production before the onset of disease symptoms (3).

Neurodegenerative diseases, such as Parkinson's disease, Alzheimer's disease and vascular dementia, are associated with oxidative damage to brain cells and the nervous system (4).

Type-II diabetes and diabetic vasculopathy or complications: Oxidative stress has emerged as a strong pathogenic co-factor in the development of long-term complications of diabetes, such as atherosclerosis, nephropathy and retinopathy (5), (6).

Respiratory disorders and lung diseases: Lung cells also abundantly express several isoforms of the Nox family, including Nox2. Enhanced ROS production contributes to the pathogenesis of many lung diseases, such as acute respiratory distress syndrome, bronchopulmonary dysplasia, emphysema, idiopathic pulmonary fibrosis, and cancer (7).

Kidney diseases: The kidney is vulnerable to oxidative damage. Although the origin of increased ROS generation in renal diseases is multi-factorial, it is well known that the kidney expresses several isoforms of the Nox family and generates ROS, and oxidative damage plays a crucial role in renal diseases and renal failure.

Inflammation and organ injury: Oxidative damage due to phagocytic Nox2 activation and inflammatory cell infiltration plays an important role in autoimmune diseases and organ injury (8), (9).

Ageing and age-related organ dysfunction: Emerging evidence has shown that ageing-associated organ disorders are related closely to increased ROS formation in various organs (10).

Thus, Nox2− derived ROS is involved in the pathogenesis of several diseases and medical conditions. It would therefore be desirable to provide Nox2 specific inhibitors as a novel approach to inhibit or reduce ROS production in cells and thereby prevent or treat numerous diseases such as cardiovascular diseases, respiratory diseases, inflammatory diseases, cancer, ageing and age related disorders, kidney diseases, neurodegenerative diseases, diabetes and complications arising from diabetes.

### SUMMARY OF THE INVENTION

The invention accordingly provides novel compounds that are inhibitors of NADPH oxidases, in particular Nox2, and that therefore find use in the treatment of a wide variety of diseases.

A first aspect of the invention provides a bi- or tri-aromatic compound according to formula (I)

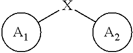

wherein A₁, A₂ and x are as defined below, as well as pharmaceutically acceptable salts, metabolites and prodrugs thereof.

A second aspect of the invention provides bi- or tri-aromatic compound according to formula (II)

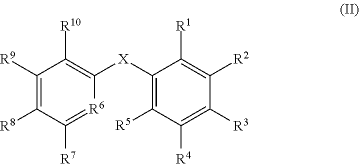

wherein R¹ to R¹⁰ and X are as defined below, as well as pharmaceutically acceptable salts, metabolites and prodrugs thereof.

A third aspect of the invention provides bi- and tri-aromatic compounds according to formula (I) or formula (II), and pharmaceutically acceptable salts, metabolites and prodrugs thereof, for use as a medicament.

A fourth aspect of the invention provides a pharmaceutical composition comprising at least one bi- or tri-aromatic compound according to the present invention, or a pharmaceutically acceptable salt, metabolite or prodrug thereof, in combination with a pharmaceutically acceptable carrier, diluent or excipient.

A fifth aspect of the invention provides a method for treating a subject suffering from a disease or condition selected from: cardiovascular diseases, respiratory diseases, inflammatory diseases, cancers, ageing and age related disorders, kidney diseases, neurodegenerative diseases, diabetes and conditions associated with diabetes. The method comprises administering to a subject in need thereof a bi- or tri-aromatic compound according to formula (I) or formula (II), wherein A₁, A₂, R¹-R¹⁰ and X are as defined below, including pharmaceutically acceptable salts, metabolites and prodrugs thereof.

A sixth aspect of the invention provides a bi- or tri-aromatic compound according to formula (I) or formula (II), wherein A₁, A₂, R¹-R¹⁰ and X are as defined below, including pharmaceutically acceptable salts, metabolites and prodrugs thereof, for use in the treatment of a disease or condition selected from: cardiovascular diseases, respiratory diseases, inflammatory diseases, cancers, ageing and age related disorders, kidney diseases, neurodegenerative diseases, diabetes and conditions associated with diabetes.

A seventh aspect of the invention provides processes for the preparation of bi- or tri-aromatic compounds according to formula (I) and formula (II), wherein A₁, A₂, R¹-R¹⁰ and X are as defined below.

### DEFINITIONS

The following definitions shall apply throughout the specification and the appended claims.

Unless otherwise stated or indicated, the term “alkyl” when used alone or in combination with other terms means a linear or branched saturated aliphatic hydrocarbon chain of 1-20 carbon atoms, including alkyl groups having the following range of carbon atoms: C1-C10, C1-C8, C1-C6 and C1-C4. Non-limiting examples of alkyl groups include methyl, ethyl, n-propyl, i-propyl, n-butyl, s-butyl, i-butyl, t-butyl, n-pentyl, 1-ethylpropyl, 2-methylbutyl, 3-methylbutyl, 2,2-dimethylpropyl, n-hexyl, 2-methylpentyl, 3-methylpentyl, 4-methylpentyl, n-heptyl, 2-methylhexyl, 3-methylhexyl, 4-methylhexyl, 5-methylhexyl, n-heptyl, n-octyl, n-nonyl, n-decyl, tetrahydrogeranyl, n-dodecyl, n-tridecyl, n-tetradecyl, n-pentadecyl, n-hexadecyl, n-octadecyl, n-nonadecyl, and n-eicosanyl and the like.

Unless otherwise stated or indicated, the term “aryl” when used alone or in combination with other terms means an unsaturated aromatic carbocyclic group of from 6 to 14 carbon atoms having a single ring or multiple condensed rings. Non-limiting examples of aryl groups include phenyl, naphthyl, indene and the like.

Unless otherwise stated or indicated, the term “arylalkyl” when used alone or in combination with other terms means an alkyl having an aryl substituent. Non-limiting examples include benzyl, phenethyl and the like.

Unless otherwise stated or indicated, the term “heterocyclic ring” when used alone or in combination with other terms means a monocyclic or bicyclic fused ring of from 4 to 14 carbon atoms wherein one or more carbon atoms is replaced with a heteroatom selected from: N, O and S, Non-limiting examples of unsaturated and partially unsaturated heterocyclic rings are: imidazole, pyrazole, oxazole, isoxazole, 1,3,4-oxadiazole, thiazole, isothiazole, pyridine, indole, thiophene, benzopyranone, thiazole, furan, quinoline, isoquinoline, pyrimidine, pyrazine, tetrazole, pyrazole, oxadidiazole, oxazine, triazine, tetrazine and the like. Non-limiting examples of saturated heterocyclic rings are: pyrrolidine, oxolane, thialane, oxazolidine, isoxazolidine, imidazolidine, pyrazolidine, thiazolidine, isothiazolidine, dioxolane, dithiolane, morpholine, trioxane and the like.

Unless otherwise stated or indicated, the term “halogen” refers to fluoro, chloro, bromo and iodo atoms.

The term “substituted” means that a H atom on a C atom is replaced with a named substituent, e.g. hydroxyl.

The term “cardiovascular disorder or disease” includes atherosclerosis, hypertension, heart failure including congestive heart failure, ischemic heart disease, coronary heart disease, peripheral artery disease, restenosis, myocardial infarction, thrombotic events including deep vein thrombosis and cardiovascular complications of Type I or Type II diabetes.

The term “respiratory disease” includes bronchial asthma, bronchitis, allergic rhinitis, adult respiratory syndrome, cystic fibrosis, lung viral infection (influenza), pulmonary hypertension, idiopathic pulmonary fibrosis and chronic obstructive pulmonary diseases (COPD).

The term “neurodegenerative disease” comprises a disease or a state characterized by a central nervous system (CNS) degeneration or alteration, especially at the level of the neurons such as Alzheimer's disease, Parkinson's disease, Huntington's disease, amyotrophic lateral sclerosis, epilepsy and muscular dystrophy.

The term “kidney disease or disorder” includes diabetic nephropathy, renal failure, glomerulonephritis, nephrotoxicity of aminoglycosides and platinum compounds and hyperactive bladder.

The term “inflammatory disease or disorder” includes inflammatory bowel disease, sepsis, septic shock, pancreatitis, shock induced by trauma, allergic rhinitis, arthritis, including rheumatoid and juvenile arthritis, psoriasis, cystic fibrosis, stroke, bronchitis, bronchiolitis, Lyme's disease, articular cell rheumatism, disorders by repetitive use (typing), hypertrophic osteoarthropathy, systemic multiple sclerosis, Crohn's disease and chronic inflammatory bowel diseases (IBD).

The term “cancer” includes colon carcinoma, pancreatic cancer, breast cancer, ovarian cancer, renal cancer, prostatic carcinoma, cervical cancer, lung cancer and bladder cancer.

The term “subject” as used herein refers to mammals, including humans, primates, animals such as cattle, horses, dogs and the like.

The term “treatment” includes prevention, reduction, amelioration or elimination of the disorder or condition.

The term “inhibitor” as used herein means a compound that inhibits completely or partially the activity of NADPH oxidase and/or inhibits or reduces the generation of ROS.

The term “pharmaceutically acceptable” means being useful in preparing a pharmaceutical composition that is generally safe, non-toxic and neither biologically nor otherwise undesirable and includes being useful for veterinary use as well as human pharmaceutical use.

Suitable pharmaceutically acceptable salts may include acid addition salts which may, for example, be formed by mixing a solution of a compound of the invention with a solution of a pharmaceutically acceptable acid such as hydrochloric acid, sulfuric acid, fumaric acid, maleic acid, succinic add, acetic acid, benzoic acid, citric acid, tartaric acid, carbonic acid or phosphoric acid. Furthermore, where the compounds of the present invention carry an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), pahnitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylat, triethiodide, undecanoate, valerate, and the like.

The term “metabolite” means any intermediate or product resulting from metabolism of a compound according to the invention.

The term “prodrug” means a functional derivative of a compound according to the invention that has a chemically or metabolically decomposable group, such as an ester or an amide, that is biotransformed in the body to form the active drug having NADPH oxidase inhibiting activity. Reference is made to Goodman and Gilman's, The Pharmacological basis of Therapeutics, 8^{th} ed., Mc-Graw-Hill, Int. Ed. 1992, “Biotransformation of Drugs”, p. 13-15.

With regard to stereoisomers, the compounds of the invention may have one or more asymmetric carbon atoms and may occur as racemates, racemic mixtures and as individual enantiomers or diastereomers. All such isomeric forms are included within the present invention, including mixtures thereof. Cis (E) and trans (Z) isomerism may also occur. The present invention includes the individual stereoisomers of the compounds of the invention and where appropriate, the individual tautomeric forms thereof, together with mixtures thereof. Separation of diastereoisomers or cis and trans isomers may be achieved by conventional techniques, e.g. by fractional crystallisation, chromatography or H.P.L.C. A stereoisomeric mixture of the compounds may also be prepared from a corresponding optically pure intermediate or by resolution, such as H.P.L.C. of the corresponding racemate using a suitable chiral support or by fractional crystallisation of the diastereoisomeric salts formed by reaction of the corresponding racemate with a suitable optically active acid or base, as appropriate.

The term “about” means plus or minus 20%, more preferably plus or minus 10%, even more preferably plus or minus 5%, most preferably plus or minus 2%.

The terms “comprises” and “comprising” mean “includes, among other things”. These terms are not intended to be construed as “consists of only”.

The reference number “LMH001” refers to the compound (2,3,-dihydroxyphenyl)methyl 4-hydroxy-3-(hydroxymethyl)benzoate.

### BRIEF DESCRIPTION OF THE FIGURES

FIG. 1 is a dose response curve showing the effect of LMH001 on NADPH—dependent O_{2.}⁻ production by cell homogenates (0.1 mg protein/well) of mouse microvascular endothelial cells (SVEC4-10). SVEC4-10 cells were cultured in 10% FCS/DMEM. Cells were harvested and homogenised. Cell homogenates were pre-incubated with LMH001 at the indicated dose for 5 min. and detected of O_{2.}⁻ production in the presence of NADPH (100 μM). O_{2.}⁻ production was measured by lucigenin (5 μM)-chemiluminescence. Results are presented as mean±SD (n=5).

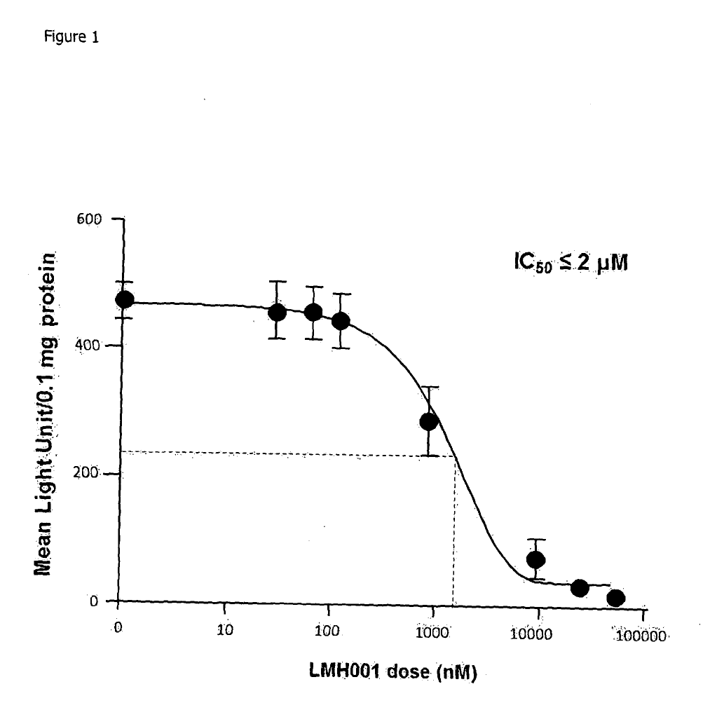

FIG. 2 is a dose response curve showing the effect of LMH001 on O_{2.}⁻ production by differentiated human neutrophil HL60 cells (5×10⁴/well). Cells were stimulated with PMA (100 ng/ml) in the presence or absence of LMH001 at the dose indicated for 5 min. O_{2.}⁻ production was measured by lucigenin (5 μM)-chemiluminescence. Results are presented as mean±SD (n=at least 5).

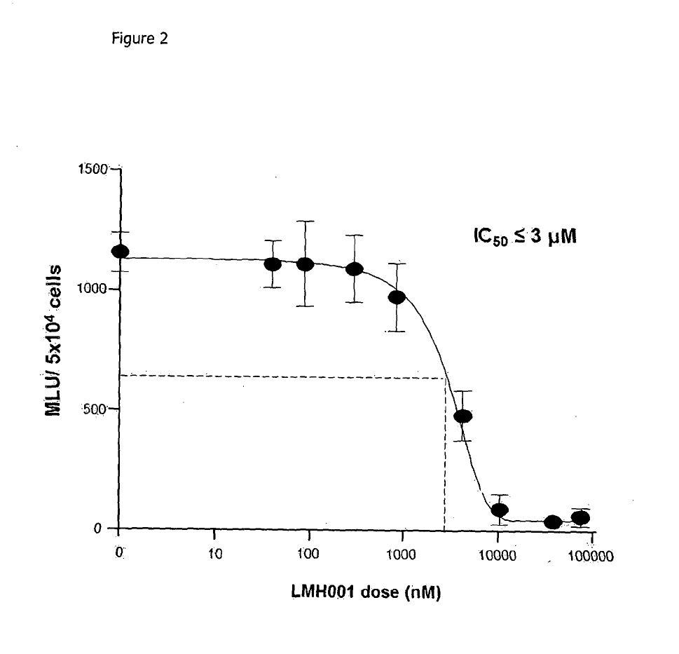

FIG. 3 is a graph showing the effect of LMH001 (2 week, IP injection) on bone marrow hematopoietic stem cell proliferation in mice. Wild-type mice (C57BL6/J) at 5 months of age were injected intra-peritoneal with LMH001 at 2.2 mg/kg/day for two weeks. Bone marrow hematopoietic stem cells were isolated and test for cell proliferation by a MTS assay kit (Promega). Compared to vehicle injected controls, LMH001 showed no toxic effect on bone marrow hematopoietic stem cell proliferation (n=8 mice/per group).

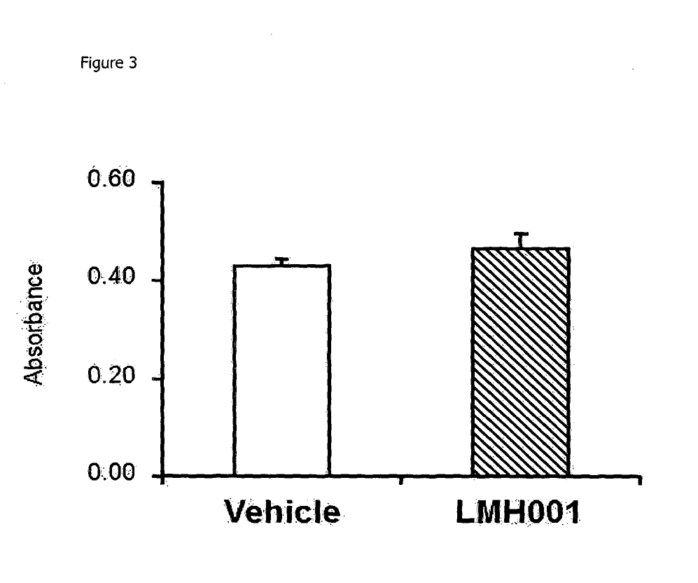

FIG. 4 shows graphs of the effect of LMH001 on superoxide dismutase (SOD)-inhibitable O_{2.}⁻ production by (A) living mouse microvascular endothelial cells (SVEC4-10) (1.5×10⁵ cells/well) and (B) mouse fibroblasts (NIH-3T3) (1.5×10⁵ cells/well), respectively, detected by means of a cytochrome c reduction assay. Results are presented as mean±SD (n=3).

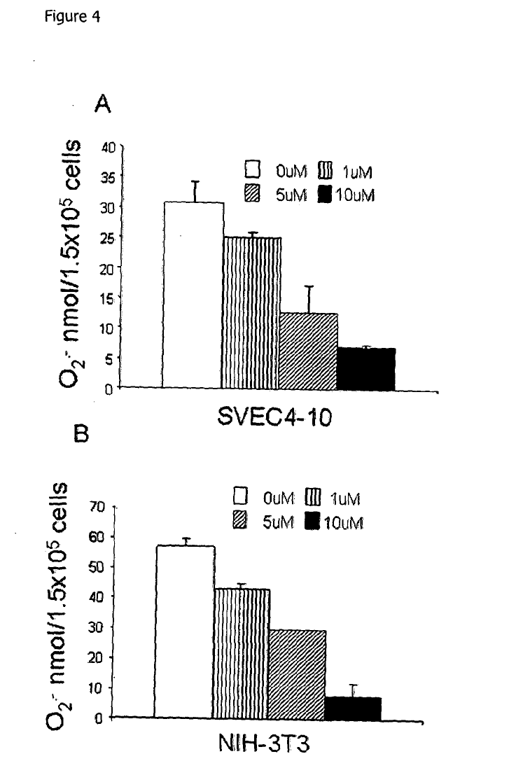

FIG. 5 shows graphs of the effect of LMH001 on O_{2.}⁻ production by different human cell types, that is, (A) Differentiated HL60 neutrophils (5×10⁴ cells/well) stimulated with PMA (100 ng/ml) and (B) Foetal lung fibroblast (IMR90) cells (5×10⁴ cells/well) cultured in 10% FCS under physiological condition. O_{2.}⁻ production was measured by lucigenin (5 μM)-chemiluminescence. The inhibitory effect of LMH001 on ROS production was expressed as a percentage of controls (100%) without LMH001 (0 μM). Results are presented as mean f SD (n=5).

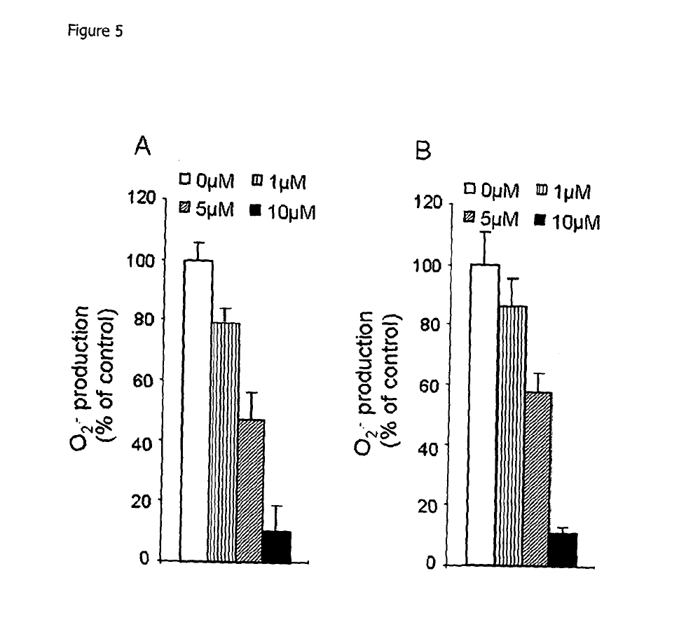

FIG. 6 is a graph showing the kinetic measurement of O_{2.}⁻ production before and after the addition of PMA (100 ng/ml) by differentiated HL60 human neutrophils. LMH001 (0-10 μM) was added 5 minutes before the measurement of O_{2.}⁻ production by lucigenin (5 μM)—chemiluminescence. Kinetic readings were taken every 2 minutes for a total of 60 minutes. PMA was added after 30 minutes of measurement and the total reading period was 60 min. Results are presented as mean f SD (n=3).

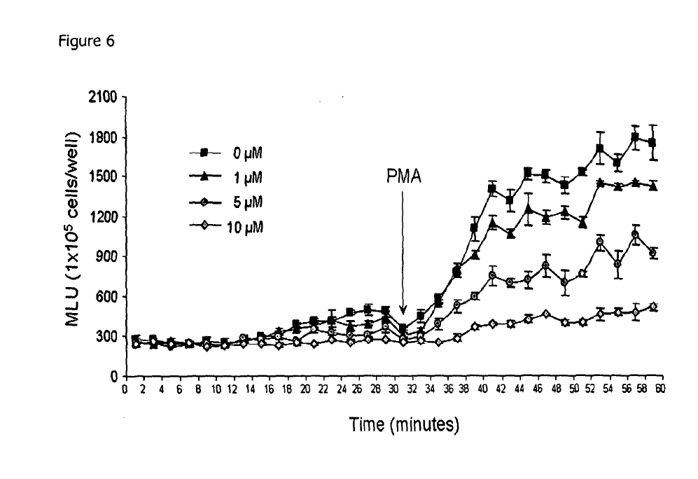

FIG. 7 is a graph showing no cytotoxicity of LMH001 on living mouse microvascular endothelial cells (SVEC4-10) and human liver hepatocyte cells (HepG2), assessed by trypan blue exclusion and viable cell number counting. Cells were cultured with LMH001 (5 and 10 μM) for 24 h. Cell viabilities of SVEC4-10 cells and HepG2 cells in the presence of LMH001 (5 and 10 μM) were expressed as % of alive cells compared to cells in the absence of LMH001 (control, 100%). Results are presented as mean±SD (n=3). There was no significant difference between cell viability of cultures in the presence (5 and 10 μM) or absence of LMH001.

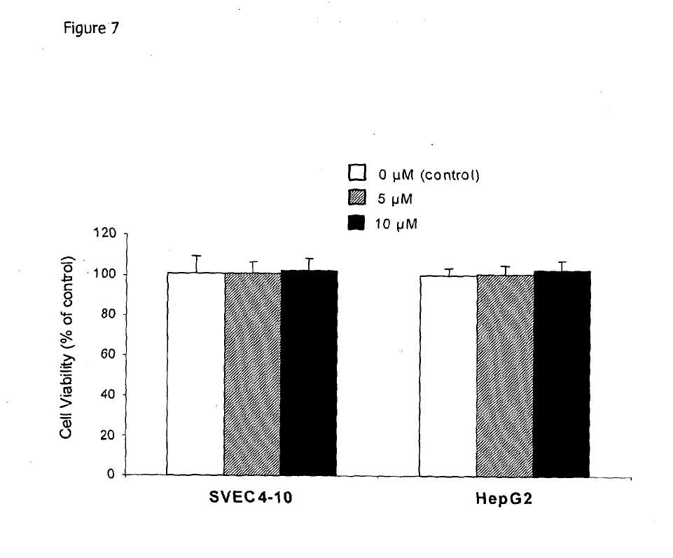

FIG. 8 is a graph showing no cytotoxicity of LMH001 on living mouse microvascular endothelial cells (SVEC4-10 cells) and human liver hepatocyte cells (HepG2 cells), assessed by means of an MTT assay. Cells were cultured with LMH001 (5 and 10 μM) for 24 h. MTT is a yellow tetrazolium reagent which can be converted by living cells to insoluble blue formazan crystal in the medium. The insoluble blue formazan crystals were then dissolved in DMSO and measured for absorbance at 570 nm by a plate reader. Higher absorbance corresponds to more living cells. Results are presented as mean±SD (n=3). There was no significant difference between cells cultured in the presence (5 and 10 μM) or absence of LMH001.

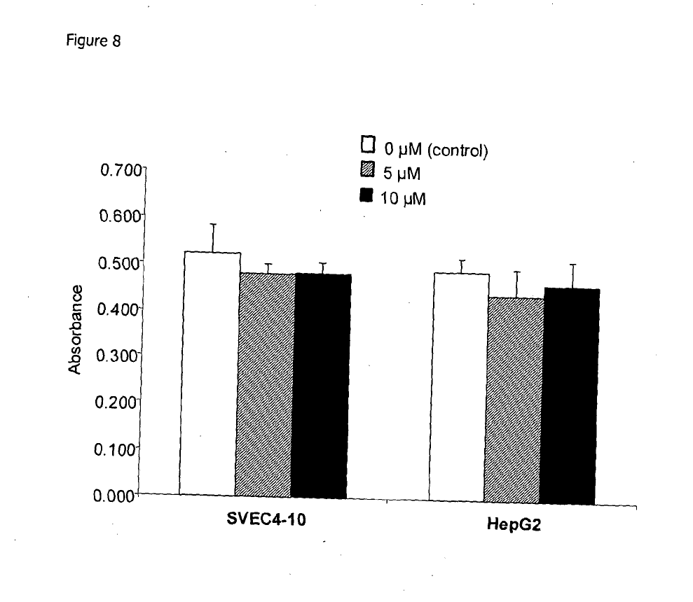

FIG. 9 shows a comparison of the inhibitory effects of different ROS generating enzyme inhibitors on acute TNFα-induced O_{2.}⁻ production by microvascular endothelial cells (SVEC4-10). SVEC4-10 cells were stimulated with TNFα (100 U/ml) for 45 min. Cell homogenates were detected for O_{2.}⁻ production in the presence of NADPH (100 μM) by lucigenin-chemiluminescence (A). Cell homogenates of TNFα-stimulated cells were pre-incubated with tiron (20 mM, superoxide scavenger), or diphenyleneiodonium (DPI, 20 μM, flavoprotein inhibitor), or LMH001 (5 μM), or apocynin (20 μM, Nox2 inhibitor) or L-NG-NitroArginine Methyl Ester (L-NAME, 100 μM, nitric oxide synthase inhibitor) or rotenone (50 μM, mitochondria complex 1 enzyme inhibitor) or oxypurinol (100 μM, xanthine oxidase inhibitor) before measuring the O_{2.}⁻ production (right panel) (n=3 independent membrane isolations). *p<0.05.

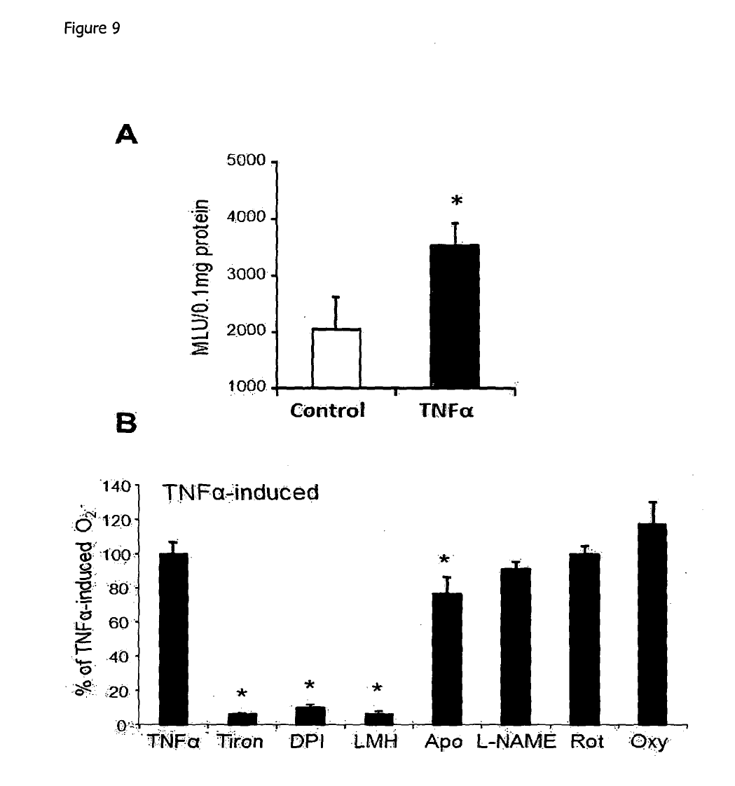

FIG. 10 is a graph showing the effects of LMH001 in culture on basal and angiotensin II induced O_{2.}⁻ production by microvascular endothelial cells (SVEC4-10). SVEC4-10 cells were cultured in medium alone (Basal) or in the presence of Angiotensin II (AngII, 200 nM) with or without LMH001 (5 μM) for 24 h. Cells were then harvested and cell homogenates were used for the measurement of NADPH-dependent O_{2.}⁻ production by lucigenin-chemiluminescence. *P<0.05 (n=3 experiments). LMH001 (24 h in culture) showed no effect on basal cell O_{2.}⁻ production, but completely inhibited AngII-induced endothelial O_{2.}⁻ production.

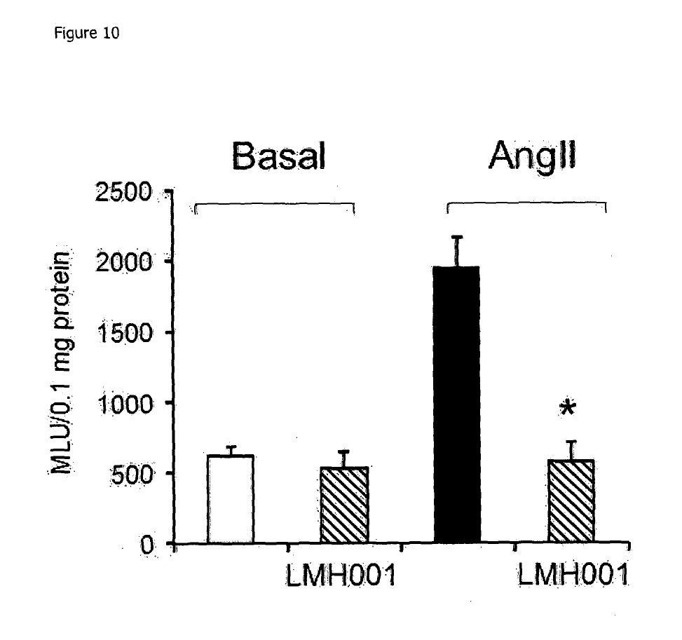

FIG. 11 is a graph showing the effects of LMH001 in culture on normal glucose level versus high glucose level-induced O_{2.}⁻ production by human pulmonary endothelial cells (HPMEC). HPMEC were cultured in the medium with normal level of glucose (5 mM) or with high level of glucose (40 mM) in the presence or absence of LMH001 (5 μM) for 24 h. Cells were harvested and cell homogenates were used for measuring NADPH (100 μM)-dependent O_{2.}⁻ production by lucigenin-chemiluminescence (n=3 experiments). Compared to control cells, LMH001 (24 h in culture) had no significant effect on cell O_{2.}⁻ production under normal glucose levels, but inhibited completely the high glucose level-induced O_{2.}⁻ production by HPMEC.

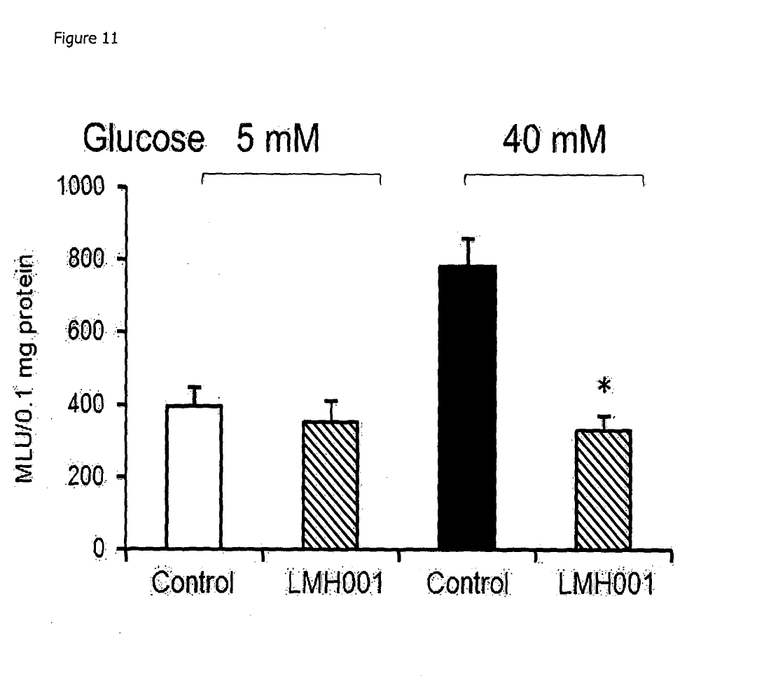

FIG. 12 shows the effects of LMH001 on EGF-induced O_{2.}⁻ production and cell proliferation in culture on human lung alveolar epithelial cancer cells (A549 cells). A549 cells were seeded at equal number the day before experiments and then stimulated with or without epithelial growth factor (EGF, 10 ng/ml) for 24 h in the presence or absence of LMH001 (5 μM). Cells were then trypsinized and counted. Cell homogenates were used for measuring O_{2.}⁻ production by lucigenin-chemiluminescence in the presence of NADPH (100 μM). Compared to control cells (medium only), LMH001 alone had no significant effect on A549 cell O_{2.}⁻ production and cell proliferation. However, LMH001 inhibited completely EGF-induced O_{2.}⁻ production and A549 cell proliferation.

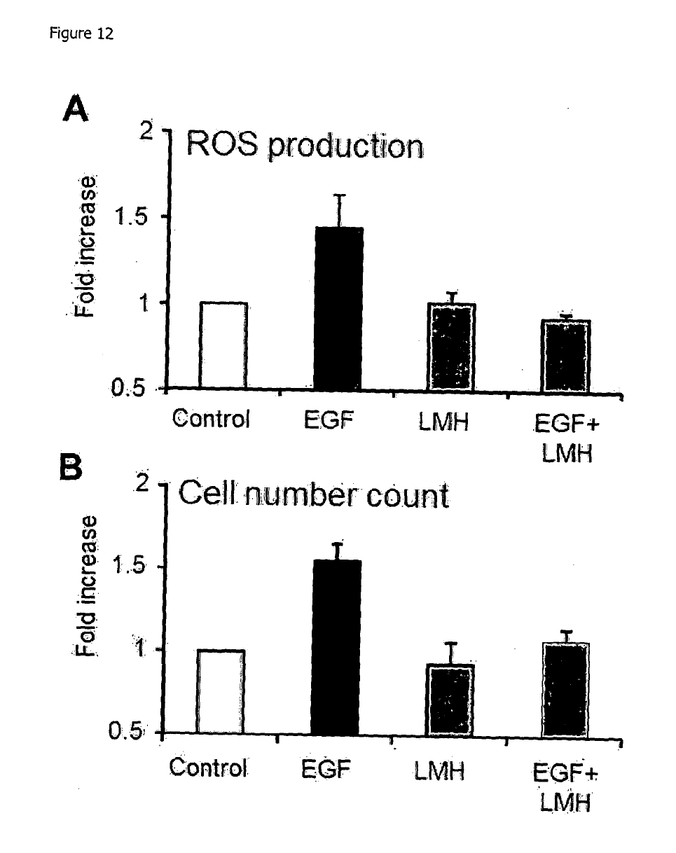

FIG. 13 shows the inhibitory effect of LHM036 on O_{2.}⁻ production by human cells. A) Differentiate human neutrophils (HL-60 cells, 5×10⁴ cells/well) were stimulated with PMA (100 ng/ml) for 5 min in the presence or absence of LMH036 at the dose indicated. O_{2.}⁻ production was measured by lucigenin-chemiluminescence. B) HPMEC were stimulated with TNFα (100 U/ml) for 45 min. Cells were immediately frozen in liquid nitrogen and homogenised. Cell homogenates (0.1 mg/well) were pre-incubated with LMH036 at the indicated dose for 5 min and measured for the O_{2.}⁻ production in the presence of NADPH (100 μM) by lucigenin-chemiluminescence.

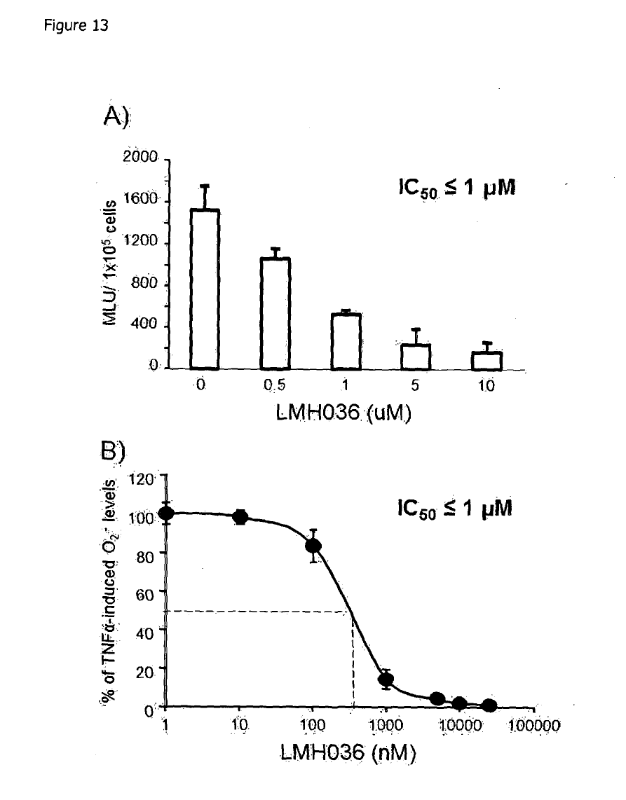

FIG. 14 shows no inhibitory effect of LHM001 on normal cell proliferation under physiological condition examined by a MTS assay. A) SVEC4-10 cells and HepG2 cells were cultured in the presence or absence of LHM001 (0-10 μM) for 24 h and tested for cell proliferation using a MTS assay kit. B) HL-60 cells were cultured in the presence or absence of LMH001 (0-10 μM), and cell proliferation was examined at 6, 12 and 24 h of culture. There was no significant effect of LMH001 up to 10 μM of concentration on normal cell proliferation under physiological condition.

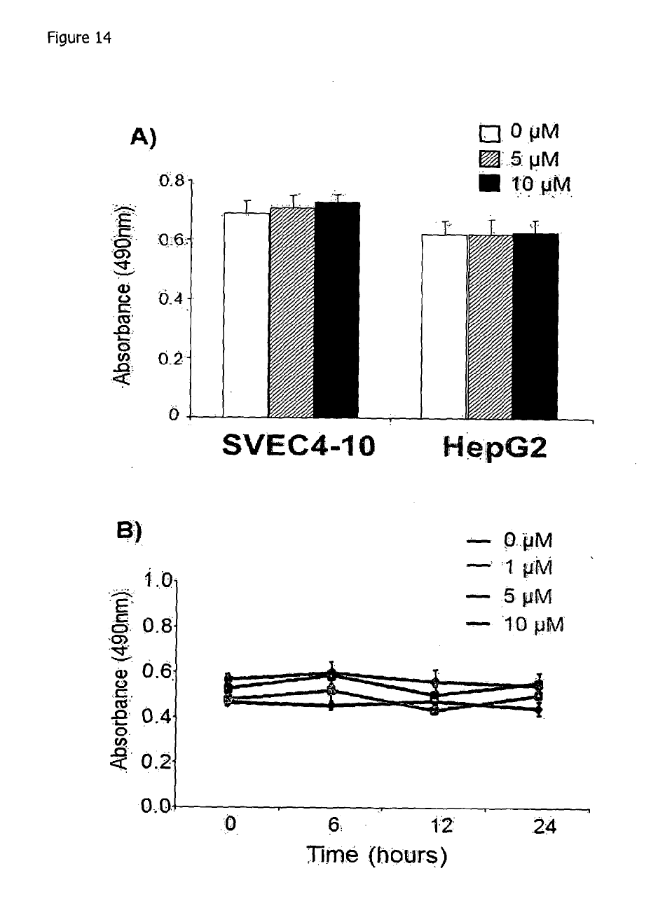

### DETAILED DESCRIPTION OF THE INVENTION

According to one aspect of the present invention, there is provided a compound of the formula (I) below.

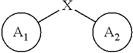

wherein
A₁ and A_{z} are independently selected from: phenyl, pyridinyl, naphthyl and quinolinyl;
A₂ is substituted by at least two OH groups; and
A₁ and A₂ are optionally substituted by one or more groups selected from: H, OR¹⁴, NR¹⁵R¹⁶, R¹⁷ and halogen; wherein
R¹⁴, R¹⁵ and R¹⁶ are each independently selected from: H and alkyl;
R¹⁷ is alkyl, aryl or arylalkyl, where each group is optionally substituted by one or more groups selected from: OR¹⁴, NR¹⁵R¹⁶ and halogen, where R¹⁴, R¹⁵ and R¹⁶ are as defined;
X is a 5 or 6-membered unsaturated, saturated or partially unsaturated carbocyclic or heterocyclic ring, wherein the heterocyclic ring has 1, 2, 3, or 4 heteroatoms selected from: O, N and S, or
X is a group of the formula below

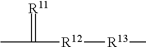

wherein

### R¹¹ is O, S or NH; and

R¹² and R¹³ are independently selected from: O, S, NH and CH₂;
in any stereochemical form, or a mixture of any stereochemical forms in any ratios;
or a pharmaceutically acceptable salt, metabolite, prodrug, or a mixture thereof.

According to another aspect of the present invention, there is provided a compound of the formula (II) below.

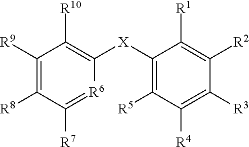

wherein
R¹, R², R³, R⁴, R⁵ and R⁷, R⁸, R⁹ and R¹⁰ are each independently selected from: H, OR¹⁴, NR¹⁵R¹⁸, R¹⁷ and halogen; where
R¹⁴, R¹⁵ and R¹⁶ are each independently selected from: H and alkyl;
R¹⁷ is alkyl, aryl or arylalkyl, where each group is optionally substituted by one or more groups selected from: OR¹⁴, NR¹⁵R¹⁶ and halogen, where R¹⁴, R¹⁵ and R¹⁶ are as defined;
or
R² and R³, together with the respective carbon atoms to which they are attached, form a 5- or 6-membered unsaturated, saturated or partially unsaturated carbocyclic or heterocyclic ring, wherein the heterocyclic ring has 1, 2, 3, or 4 heteroatoms selected from: O, N and S, and wherein the carbocyclic or heterocyclic ring is optionally substituted by one or more groups selected from: OR¹⁴, NR¹⁵R¹⁶, R¹⁷ and halogen, where R¹⁴, R¹⁵, R¹⁶ and R¹⁷ are as defined;
or
R³ and R⁴, together with the respective carbon atoms to which they are attached, form a 5- or 6-membered unsaturated, saturated or partially unsaturated carbocyclic or heterocyclic ring, wherein the heterocyclic ring has 1, 2, 3, or 4 heteroatoms selected from: O, N and S, and wherein the carbocyclic or heterocyclic ring is optionally substituted by one or more of groups selected from: OR¹⁴, NR¹⁵R¹⁶, R¹⁷ and halogen, where R¹⁴, R¹⁵, R¹⁶ and R¹⁷ are as defined;
or
R⁸ and R⁹, together with the respective carbon atoms to which they are attached, form a 5- or 6-membered unsaturated, saturated or partially unsaturated carbocyclic or heterocyclic ring, wherein the heterocyclic ring has 1, 2, 3, or 4 heteroatoms selected from: O, N and S, and wherein the carbocyclic or heterocyclic ring is optionally substituted by one or more groups selected from: OR¹⁴, NR¹⁵R¹⁶ R¹⁷ and halogen, where R¹⁴, R¹⁵, R¹⁶ and R¹⁷ are as defined;
or
R⁹ and R¹⁰, together with the respective carbon atoms to which they are attached, form a 5- or 6-membered unsaturated, saturated or partially unsaturated carbocyclic or heterocyclic ring, wherein the heterocyclic ring has 1, 2, 3, or 4 heteroatoms selected from: O, N and S, and wherein the carbocyclic or heterocyclic ring is optionally substituted by one or more groups selected from: OR¹⁴, NR¹⁵R¹⁶, R¹⁷ and halogen, where R¹⁴, R¹⁵, R¹⁶ and R¹⁷ are as defined;

### R⁶ is N or CH;

X is a 5 or 6-membered unsaturated, saturated or partially unsaturated carbocyclic or heterocyclic ring, wherein the heterocyclic ring has 1, 2, 3, or 4 heteroatoms selected from: O, N and S, or
X is a group of the formula below

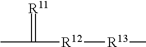

wherein

### R¹¹ is O, S or NH; and

R¹² and R¹³ are independently selected from: O, S, NH and CH₂;
in any stereochemical form, or a mixture of any stereochemical forms in any ratios; or a pharmaceutically acceptable salt, metabolite, prodrug, or a mixture thereof.

In one embodiment of the compound of the formula (I), R¹, R², R³, R⁴, R⁵, R⁷, R⁸, R⁹ and R¹⁰ are each independently selected from: H, OH, alkyl and aralkyl, where the alkyl and arylalkyl groups are optionally independently substituted by 1, 2 or 3 groups selected from: OH and hydroxyalkyl;

or
R² and R³, together with the respective carbon atoms to which they are attached, form a 6-membered unsaturated carbocyclic ring, optionally substituted by one or more groups selected from: OH, alkyl and hydroxyalkyl;
or
R³ and R⁴, together with the respective carbon atoms to which they are attached, form a 6-membered unsaturated carbocyclic ring, optionally substituted by one or more groups selected from: OH, alkyl and hydroxyalkyl;
or
R⁸ and R⁹, together with the respective carbon atoms to which they are attached, form a 6-membered unsaturated or partially unsaturated carbocyclic or heterocyclic ring, wherein the heterocyclic ring has 1, 2 or 3 heteroatoms selected from: O, N and S, and wherein the carbocyclic or heterocyclic ring is optionally substituted by one or more groups selected from: OH, alkyl, hydroxyalkyl, benzyl, optionally substituted by 1 or 2 OH and phenyl, optionally substituted by 1 or 2 OH;
or
R⁹ and R¹⁰, together with the respective carbon atoms to which they are attached, form a 6-membered unsaturated carbocyclic ring, optionally substituted by one or more groups selected from: OH, alkyl and hydroxyalkyl;

### R⁶ is N or CH;

X is a 5-membered unsaturated or partially unsaturated heterocyclic ring having 1, 2 or 3 heteroatoms selected from: O, N and S, or
X is a group of the formula below

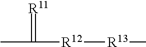

wherein

### R¹¹ is O or NH;

### R¹² is O, NH or CH₂, and

### R¹³ is CH₂;

in any stereochemical form, or a mixture of any stereochemical forms in any ratios; or a pharmaceutically acceptable salt, metabolite, prodrug, or a mixture thereof.

In another embodiment, the invention provides a compound of the formula (III) below

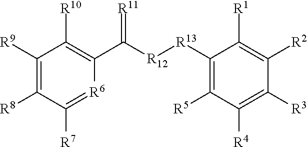

wherein R¹ to R⁵ and R⁷, R⁸ and R¹⁰ are independently selected from: H, OH, alkyl and hydroxyalkyl;
R⁹ is selected from: H, OH, alkyl and benzyl, where the alkyl and benzyl groups are optionally substituted by 1 or 2 OH;
or
R² and R³, together with the respective carbon atoms to which they are attached, form a 6-membered unsaturated carbocyclic ring, optionally substituted by one or more groups selected from: OH, alkyl and hydroxyalkyl;
or
R³ and R⁴, together with the respective carbon atoms to which they are attached, form a 6-membered unsaturated carbocyclic ring, optionally substituted by one or more of the groups: OH, alkyl and hydroxyalkyl;
or
R⁸ and R⁹, together with the respective carbon atoms to which they are attached, form a 6-membered unsaturated or partially unsaturated carbocyclic or heterocyclic ring, wherein the heterocyclic ring has 1 or 2 heteroatoms selected from: O, N and S, and wherein the carbocyclic or heterocyclic ring is optionally substituted by one or more of the groups: OH, alkyl and hydroxyalkyl, benzyl, optionally substituted by 1 or 2 OH and phenyl, optionally substituted by 1 or 2 OH;

### R⁶ is N or CH;

### R¹¹ is O or NH;

### R¹² is O, NH or CH₂; and

### R¹³ is CH₂;

in any stereochemical form, or a mixture of any stereochemical forms in any ratios; or a pharmaceutically acceptable salt, metabolite, prodrug, or a mixture thereof,

In another embodiment, the invention provides a compound of the formula (IV) below

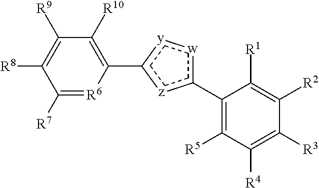

wherein
R¹ to R⁵ and R² to R¹⁰ are independently selected from: H, OH, alkyl and hydroxyalkyl;
or
R⁸ and R⁹, together with the respective carbon atoms to which they are attached, form a 6-membered unsaturated carbocyclic ring, optionally substituted by one or more groups selected from: OH, alkyl and hydroxyalkyl;
or
R⁹ and R¹⁰, together with the respective carbon atoms to which they are attached, form a 6-membered unsaturated carbocyclic ring, optionally substituted by one or more groups selected from: OH, alkyl and hydroxyalkyl;

### R⁶ is N or CH;

the atoms z, y and w are independently selected from: C, O, N and S;
the dashed line represents an optional bond;
in any stereochemical form, or a mixture of any stereochemical forms in any ratios; or a pharmaceutically acceptable salt, metabolite, prodrug, or a mixture thereof.

Any known compound having a structural formula identical to any one of the compounds covered by formula (I), (II), (III) or (IV) is hereby explicity disclaimed per se.

In particular embodiments of the compound of the formula (I), (II), (III) or (IV), R¹ and R² are OH and R³, R⁴ and R⁵ are H; or R⁴ and R⁵ are OH and R¹, R² and R³ are H.

In another particular embodiment of the compound of the formula (I), (II), (III) or (IV), R⁸ and R⁹ are independently selected from: methyl, hydroxymethyl and OH; and R⁷ and R¹⁰ are H.

In another embodiment of the compound of the formula (I), (II) or (III), R¹¹ is O, R¹² is O and R¹³ is CH₂.

In another embodiment of the compound of the formula (I) or (II), X is a 5-membered unsaturated or partially unsaturated heterocyclic ring having a N and a O heteroatom.

In another embodiment of the compound of the formula (I), (II), (III) or (IV), R² and R³, together with the respective carbon atoms to which they are attached, form a 6-membered unsaturated carbocyclic ring, optionally substituted by 1, 2 or 3 OH.

Compounds of the present invention include in particular those selected from:
2,3-dihydroxyphenyl)methyl 4-hydroxy-3-(hydroxymethyl)benzoate:
3-(2,3-dihydroxyphenyl)-1-(4-hydroxy-3-(hydroxymethyl)phenyl)propan-1-one;
3-(2,3-dihydroxyphenyl)-1-(3,4-dihydroxyphenyl)propan-1-one;
N-(2,3-dihydroxybenzyl)-4-hydroxy-3-(hydroxymethyl)benzamide;
N-(2,3-dihydroxybenzyl)-3,4-dihydroxybenzamide;
N-(2,3-dihydroxybenzyl)-3-hydroxy-4-(hydroxymethyl)benzamine;
2,3-dihydroxybenzyl 4-hydroxy-3-(hydroxymethyl)benzimine;
2,3-dihydroxybenzyl 4-hydroxy-3-(3-hydroxybenzyl)benzoate;
2,3-dihydroxybenzyl 3-benzyl-4-hydroxybenzoate;
2,3-dihydroxybenzyl 3-(hydroxymethyl)-4-(hydroxymethyl)benzoate;
2,3-dihydroxybenzyl 3,4-dihydroxybenzoate;
2,3-dihydroxybenzyl 3-hydroxy-4-methylbenzoate;
2,4-dihydroxybenzyl 3-hydroxy-4-(hydroxymethyl)benzoate;
2,3-dihydroxybenzyl 2-napthoate;
2,3-dihydroxybenzyl 6-hydroxy-7-(4-hydroxyphenyl)-2-napthoate;
2,3-dihydroxybenzyl 6-hydroxy-7-phenyl-2-napthoate;
2,3-dihydroxybenzyl 6-hydroxy-7-methyl-2-napthoate;
2,3-dihydroxybenzyl 6-hydroxy-7-(hydroxymethyl)-2-napthoate;
2,3-dihydroxybenzyl 6,7-dihydroxy-2-napthoate;
2,3-dihydroxybenzyl 7-hydroxy-2-napthoate;
2,3-dihydroxybenzyl 6,8-dihydroxyisoquinoline-3-carboxylate; and
2,3-dihydroxybenzyl 8-hydroxy-6-(hydroxymethyl)isoquinoline-3-carboxylate;
3,8-dihydroxynapthalene-2-yl)methyl 4-hydroxy-3-(hydroxymethyl)benzoate;
2,3-dihydroxybenzyl 1,5-dihydroxyisoquinoline-6-carboxylate; and
7,8-dihydroxynapthalene-2-yl)methyl 4-hydroxy-3-(hydroxymethyl)benzoate;

Compounds of the present invention further include in particular those selected from:
3-(2-(4-hydroxy-3-(hydroxymethyl)phenyl)-1H-oxazol-5-yl)benzene-1,2-diol;
4-(5-(2,3-dihydroxyphenyl)-4,5dihydrooxazol-2-yl)naphthalene-2,8-diol;
3-(2-(6-hydroxy-4-methylnapthalen-1-yl)oxazol-5-yl)benzene-1,2-diol;
3-(2-(6-hydroxy-4-(hydroxymethyl)napthalen-1-yl)oxazol-5-yl)benzene-1,2-dial;
3-(2-(napthalen-1-yl)oxazol-5-yl)benzene-1,2-diol;
3-(2-(6-hydroxy-7-methylnapthalen-1-yl)benzene-1,2-diol;
3-(2-(6-hydroxy-7-(hydroxymethyl)napthalen-1-yl)oxazol-5-yl)benzene-1,2-did;
5-(5-(2,3-dihydroxyphenyl)oxazol-2-yl)naphthalene-2,3-diol;
3-(2-(7-hydroxy-6-methylnapthalen-1-yl)oxazol-5-yl)benzene-1,2-diol;
3-(2-(4-hydroxy-3-(hydroxymethyl)phenyl)oxazol-5-yl)benzene-1,2-diol;
3-(2-(napthalen-1-yl)oxazol-5-yl)benzene-1,2-diol;
3-(2-(6-hydroxy-7-(hydroxymethyl)napthalen-2-yl)oxazol-5-yl)benzene-1,2-diol;
6-(5-(2,3-dihydroxyphenyl)oxazol-2-yl)napthalene-2,3-diol; and
3-(2-(7-hydroxynapthalen-2-yl)oxazol-5-yl)benzene-1,2-diol.

In another embodiment, a preferred compound of the formula (III) comprises one or more of the characteristics identified below:
a molecular formula of C₁₅H₁₄O₆;
a molecular weight of 290.27;

In another embodiment, a preferred compound of the formula (IV) comprises one or more of the characteristics identified below:
a molecular formula of C₁₆H₁₃NO₅;
a molecular weight of 299.28;

In another embodiment, the invention provides a compound according to formula (I), (II), (III) or (IV), or a pharmaceutically acceptable salt, metabolite, prodrug, or a mixture thereof, for use as a medicament.

In another embodiment, the invention provides the use of a compound according to formula (I), (II) (III) or (IV), or a pharmaceutically acceptable salt, metabolite, or prodrug thereof, preferably a therapeutically acceptable amount thereof, in the manufacture of a medicament for the treatment of a disease or condition selected from: cardiovascular diseases, respiratory diseases, inflammatory diseases, cancer, ageing and age related disorders, kidney diseases, neurodegenerative diseases, diabetes and conditions associated with diabetes.

In another embodiment, the invention provides a compound according to formula (I), (II) (III) or (IV), or a pharmaceutically acceptable salt, metabolite, prodrug, or a mixture thereof, for use in the treatment of a disease or condition selected from: cardiovascular diseases, respiratory diseases, inflammatory diseases, cancer, ageing and age related disorders, kidney diseases, neurodegenerative diseases, diabetes and conditions associated with diabetes.

In another embodiment, the invention provides a method for treating a subject suffering from a disease or condition selected from: cardiovascular diseases, respiratory diseases, inflammatory diseases, cancer, ageing and age related disorders, kidney diseases, neurodegenerative diseases, diabetes and conditions associated with diabetes, the method comprising administering to the subject in need thereof a compound of the formula (I), (II), (III) or (IV), or a pharmaceutically acceptable salt, metabolite, prodrug, or a mixture thereof.

In one embodiment, the compounds according to the invention are for use in the treatment of a disease or medical condition in an animal, preferably a mammal, more preferably a human. In another embodiment, the compounds according to the invention are for use in the treatment of a disease or medical condition in a non-human mammal, such as a dog, cat, horse, etc. The compounds according to the present invention therefore have application in both human and veterinary medicine.

In another embodiment, the invention provides a pharmaceutical composition comprising a compound according to the present invention, or a salt, metabolite, or prodrug thereof, in combination with a pharmaceutically acceptable carrier, diluent or excipient thereof.

The pharmaceutical composition may further comprise one or more other therapeutic agents.

In one embodiment, a therapeutic agent, other than a compound of the invention, may be administered concurrently with a compound of the invention. For example, in the treatment of cancer, a compound of the invention may be administered in combination with a co-agent used in conventional chemotherapy directed against solid tumors. The different therapeutic agents may be administered sequentially, separately or simultaneously.

In another aspect of the invention, there is provided a process for the preparation of a compound of the formula (I), (II) or (III), comprising reacting a compound of the formula (V) below

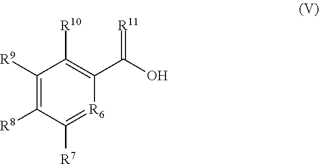

wherein R⁶ to R¹¹ are as defined, optionally wherein any one or more of the groups R⁷ to R¹⁰ are protected, with a compound of the formula (VI) below

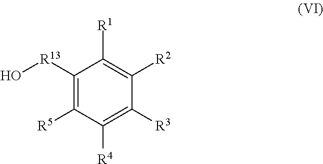

wherein R¹ to R⁵ and R¹³ are as defined, optionally wherein any one or more of the groups R¹ to R⁵ are protected, to form a compound of the formula (III), after removal of any protecting groups, wherein R¹² is O, and R¹ to R¹¹ and R¹³ are as defined, and, optionally, converting the compound into a pharmaceutically acceptable salt. The protection and de-protection of functional groups are routine procedures to the person skilled in the art.

According to a further aspect of the invention, there is provided a process for the preparation of a compound of the formula (X) below

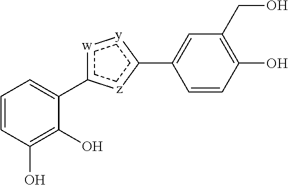

wherein w, y and z are as defined, and the dashed line represents an optional bond, comprising reacting a compound of the formula (XI)

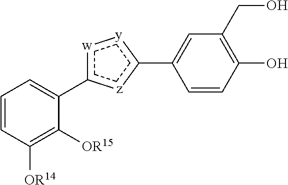

wherein R¹⁴ and R¹⁵ are protective groups, with dry 10% Pd/carbon, where “dry” means substantially no water or less than about 5% water, in an organic solvent, optionally in the presence of triethylamine, to form the compound of the formula (X), and, optionally, converting the resultant compound into a pharmaceutically acceptable salt.

The invention further provides intermediate compounds of formula (V), (VI) and (VIII) below:

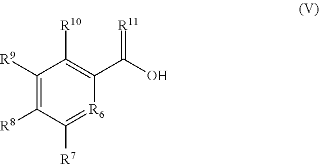

wherein R⁶ to R¹¹ are as defined in the detailed description;

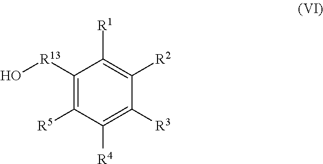

wherein R¹ to R⁵ and R¹³ are as defined in the detailed description,

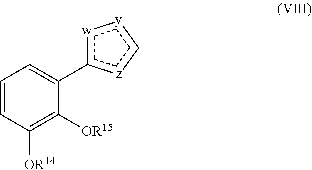

where R¹⁴, R¹⁵, w, y and z are as defined in the detailed description, and the dashed line represents an optional bond.

According to a further aspect of the invention, there is provided a process for the preparation of an intermediate compound of the formula (VII) below

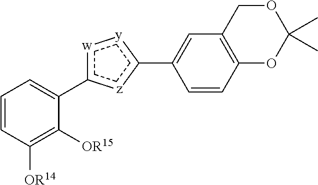

where R¹⁴ and R¹⁵ are protective groups, and w, y and z are independently selected from: C, O, N and S, and the dashed line represents an optional bond,
comprising reacting a compound of the formula (VIII) below

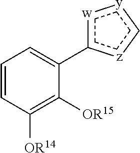

where R¹⁴, R¹⁵, w, y, z and the dashed line are as defined, with a compound of the formula (IV) below

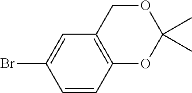

in the presence of Pd(Ph₃P)₄ and ^{t-}BuOLi in an organic solvent, preferably 1, 4-dioxane, at a temperature of about 80-160° C., preferably about 100-150° C., more preferably about 120° C., to form the intermediate compound of the formula (VII), optionally converting the compound of the formula (VII) into a compound of the formula (XI).

Insofar as the compounds according to the invention are present as stereoisomers, they can be separated using known methods, for example by means of separation using a chiral column.

The chemical structures of the compounds according to the invention were determined by the use of mass spectral and ¹H NMR and ¹³C NMR spectral measurements.

As noted above, it is preferred that a therapeutically effective amount of the compound of the formula (I), (II), (III) or (IV) in any stereochemical form, or a mixture of any stereochemical forms in any ratios, or a pharmaceutically acceptable salt, metabolite, or prodrug thereof, is present or is used in the above aspects of the invention.

The compounds according to the invention may be incorporated into pharmaceutical compositions suitable for administration. Such compositions typically comprise at least one compound of the invention and at least one pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic add; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that syringability exists. It must be stable under the conditions of manufacture, transfer and storage. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum mono stearate and gelatin.

Sterile injectable solutions can be prepared by incorporating the active compound (e.g., a Nox2 inhibitor according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray of liquid, or powdered or formulated inhibitor (e.g. within liposomes as stated below) from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds can be formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred.

While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

When using the compounds according to the present invention, the dose can vary within wide limits and, as is customary and is known to the physician, is to be suited to the individual conditions in each individual case. It depends, for example, on the nature and severity of the disease to be treated, on the mode of administration, or on whether an acute or chronic condition is treated or whether prophylaxis is carried out. An appropriate dosage can be established using clinical approaches well known in the medical art. In general, the daily dosage for achieving the desired results in an adult weighing about 75 kg is from about 0.01 to about 100 mg/kg, preferably from about 0.1 to about 50 mg/kg, in particular from about 0.1 to about 10 mg/kg.

Within this specification embodiments have been described in a way which enables a clear and concise specification to be written, but it is intended and will be appreciated that embodiments may be variously combined or separated without parting from the invention.

The invention will now be further illustrated by the non-limiting experimental procedures and results detailed below.

Schemes 1 and 2 outline a multi-step two-part convergent synthesis showing typical or preferred experimental conditions for the preparation of the compounds LMH001 and LMH026, respectively. It will be apparent that other experimental conditions (i.e., reaction temperatures, time, moles of reagent solvents, etc.) can also be used for the preparation of LMH001 and LMH036 and that the experimental conditions and procedures can be suitably modified for the preparation of other compounds falling within the formula (I).

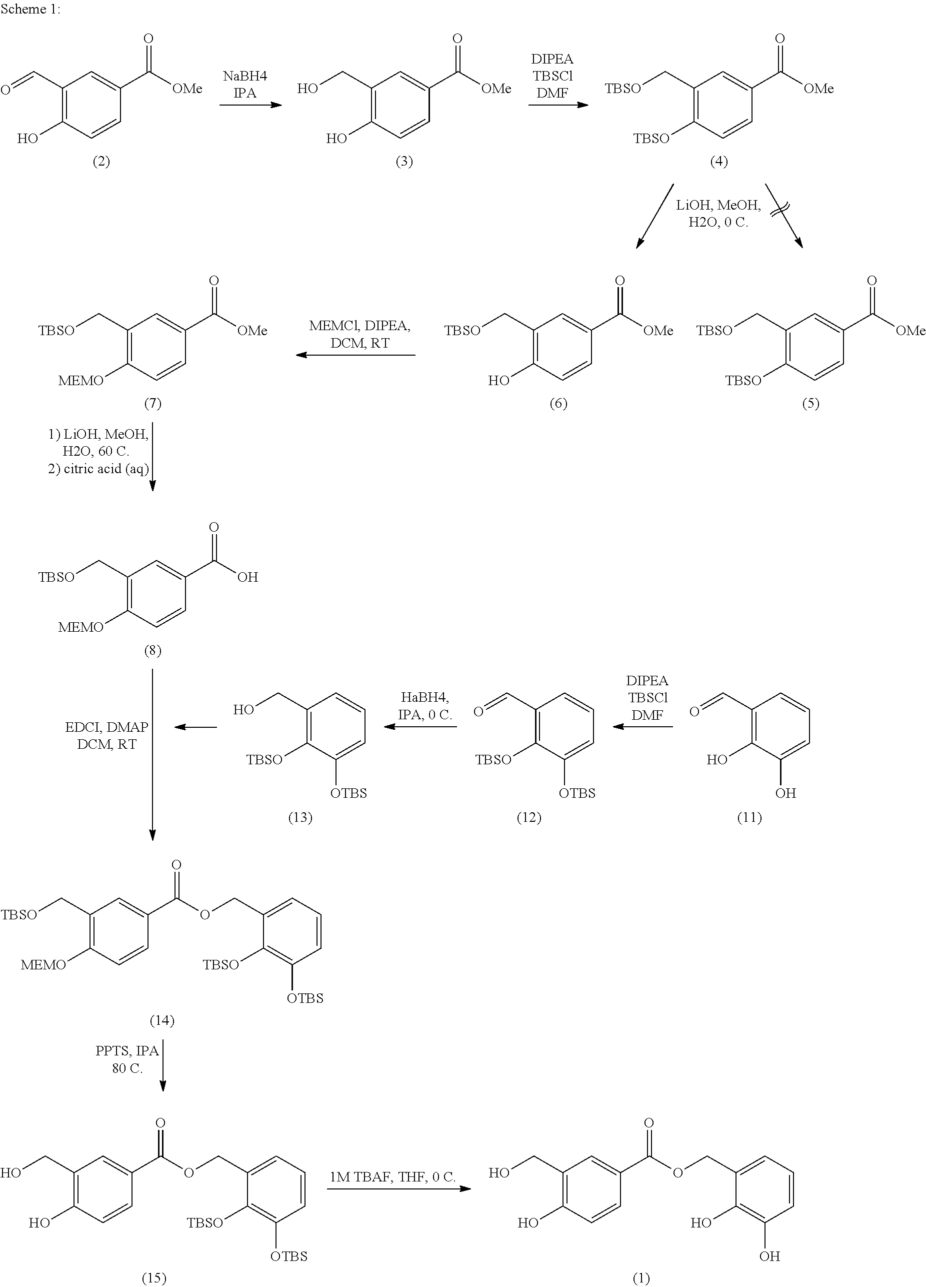

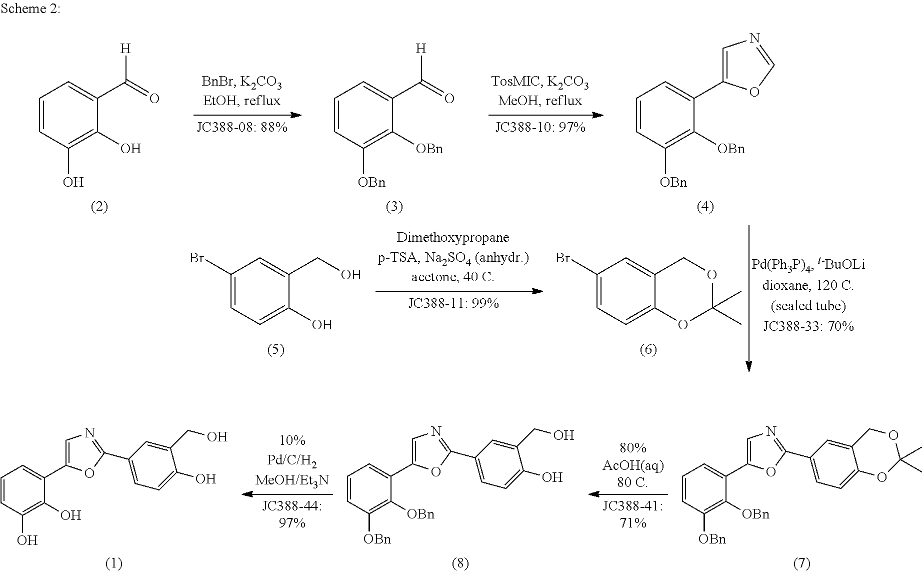

Explanation of Abbreviations used in Schemes 1 and 2.
aq (aqueous), C (° celcius), RT (room temperature), DCM (dichloromethane), DIPEA (di-isopropyl ethylamine), DMSO (dimethyl sulphoxide), DMF (N,N-dimethylformamide), MEM (2-methoxyethoxymethyl), TFA (trifluoroacetic acid), THF (tetrahydrofuran), NaBH₄ (sodium borohydride), IPA (isopropyl alcohol), TBS (tert-butyl dimethylsilyl), LiOH (lithium hydroxide), MeOH (methanol), H₂O (water), ED (carbondiimide), DMAP (4-Dimethylaminopyridine), PPTS (Pyridinium p-toluenesulfonate), TBAF (Tetra-n-butylammonium fluoride), TosMIC (toluenesulfonylmethyl-isocyanide), p-TSA (p-toluene sulfonic acid), AcOH (acetic acid), BnBr (benzyl bromide), Et₃N (triethylamine).

### Scheme 1

### Preparation of (2,3-dihydroxyphenyl)methyl 4-hydroxy-3-(hydroxymethyl)benzoate (LMH001)

LMH001 is made from commercially available starting materials according to the steps outlined in Scheme 1. In one step of compound synthesis, the commercially available aldehyde of formula (2) was reduced with sodium borohydride under standard conditions to give the diol of formula (3). The hydroxyl groups of the diol were then protected using the silyl ethers TBS to provide the bis-silyl ether of formula (4). Attempted saponification of the ester using LiOH removed one phenolic TBS group to give the compound of formula (6). The free hydroxyl group in the compound of formula (6) was re-protected as a MEM ether to give the orthogonally protected diol of formula (7). Saponification of the compound of formula (7) produced the acid of formula (8), which was ready for coupling with the product of the other step for the full synthesis of the above identified compound.

In a separate step of compound synthesis, a commercially available dihydroxybenzaldehyde of formula (11) is protected by bis-silylation to produce the protected diol of formula (12). The aldehyde functional group was then reduced with sodium borohydride to produce the benzyl alcohol of formula (13). The acid compound of formula (8) and the alcohol compound of formula (13) are then reacted in a diimide-mediated coupling to give the ester compound of formula (14). The final steps involved the removal of the MEM ether by treatment with PPTS in refluxing IPA and the cleavage of the phenolic silyl ethers using TBAF in THF to produce the compound 2,3-dihydroxyphenyl)methyl 4-hydroxy-3-(hydroxymethyl)benzoate of formula (1).

### Scheme 2

### Preparation of 3-{2-[4-hydroxy-3-(hydroxymethyl)phenyl]-1,3-oxazol-5-yl}benzene-1,2-diol (LMH036)

No published synthesis of LMH036 (1) exists and so a route was proposed as part of the evaluation process. This proposal is shown in Scheme 2.

2.1: In the first step, commercially available 2,3-dihydroxybenzaldehyde (2) was protected as the bis-benzylated derivative (3) using standard conditions. The bis-benzylated derivative (3) was obtained in good yield and high purity after recrystallisation.

2.2: The second step was the conversion of an aldehyde (3) to an oxazole (4) using TosMIC under standard conditions (17). The reaction furnished a pure product in quantitative yield after trituration from water and thorough drying.

2.3: Having secured one suitably protected fragment (4), we now required the other partner for the key coupling reaction. Our initial proposal was to bis-benzylate the commercially available bromo-diol (5) and use that in the coupling because the resulting product would only require a global de-benzylation to give (1). However, a concern was raised that deprotection of the benzylic hydroxyl function could result in cleavage of the wrong bond and require a re-start. For this reason, it was decided to make the acetonide (6) instead. This was duly achieved in quantitative yield using standard conditions.

2.4: Much experimentation was required before the optimum conditions for the coupling reaction were found. Initially, the conditions reported by Besselievre et al., (17) were employed using K₂CO₃, Pd(OAc)₂, and CuI in DMF at 150° C. (sealed tube). This method did produce some of the desired product (7) (20%) along with a lot of unreacted starting material. When the reaction was run for a longer time to try and force it to completion, thin layer chromatography indicated that it had stalled. However, further investigation revealed that in fact what happens is that the oxazole homo-dimer (9) starts to form, and because it runs at the same Rate of flow (Rf) as (4), the reaction only appears to have of stalled.

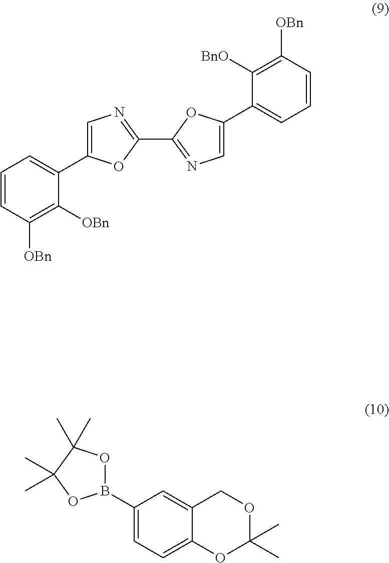

Besselievre's conditions were modified to try and overcome this problem (e.g. PCy3 ligand added, Cs₂CO₃ used as base, the reaction was carried out at atmospheric pressure) but no improvement was evident.

Yang et al., (19) reported the coupling of aryl boronate esters with oxazoles under an oxidative atmosphere. Thus, the boronate ester (10) was prepared (in one step from (6)) and an attempt was made to couple it to (4) under the reported conditions (^{t-}BuONa, CuCl, DMF, O₂ at 40° C.). Unfortunately, this gave almost exclusively (9) and no sign of (7).

Since the boronate (10) was available, it was tried as a coupling partner under Piguel's conditions (previously employed with the bromide (6)) but, again, the result was exclusive formation of (9).

Besselievre et al., (17) describes the coupling of oxazoles with aryl bromides using the relatively simple combination of Pd(Ph₃P)₄ and ^{t-}BuOLi in 1,4-dioxane at 120° C. (sealed tube). Surprisingly, when these conditions were applied to our substrates the coupling proceeded to give (7) in good yield and high purity after chromatography.

2.5: With (7) in hand, attention turned to the removal of the protecting groups. The acetonide was cleaved first to give the diol (8) using a method reported (20) for a similar substrate.

On the large scale run, the product (8) precipitated from the reaction mixture in acceptable yield and good purity.

2.6: The final step required the debenzylation of (8) to give the desired product LMH036 (1). The reaction was trialled using 10% Pd/carbon (50% wet with water for safety) in ethanol under a hydrogen balloon. Thin layer chromatography indicated complete consumption of starting material after one hour with formation of two new spots. It was assumed that these were the mono and bis-debenzylation products and the reaction was left to run for longer. Eventually, after 24 hours, only one spot remained and this was isolated. However, analysis showed that it was not (1) but rather the de-oxygenated compound (11) (Scheme 2). It was proposed that this resulted from an acid catalysed process that gave the intermediate quinone-methide (12) which was subsequently hydrogenated to (11).

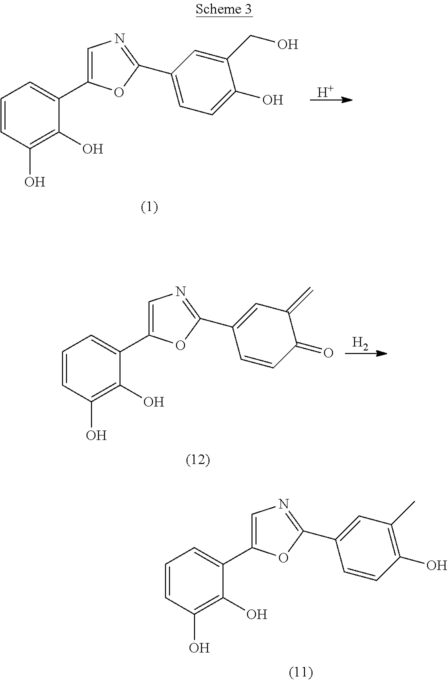

To try to remove the source of the acid, dry instead of wet 10% Pd/C was tried together with an equivalent of triethylamine in situ. This had the desired effect and the de-benzylation reaction gave LMH036 (1) in quantitative yield and sufficient purity (97%) after simple trituration from DCM.

### Structure of LMH001

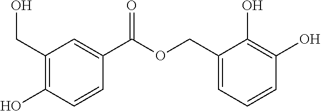 [SMILES: O=C(OCc1cccc(O)c1O)c1ccc(O)c(CO)c1]

The structures of further compounds according to the invention, their binding energies (BE) and calculated IC₅₀s are listed in the following Table 3:

TABLE 3

| | | | BE | IC₅₀ |
|---|---|---|---|---|
| ID | Name | Structure | KJ/mol | (μM) |
| LMH002 | 3-(2-(4-hydroxy-3- (hydroxymethyl)phenyl)-1H- oxazol-5-yl)benzene-1,2-diol | 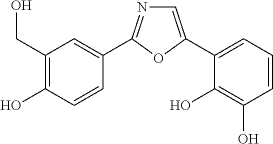 [SMILES: OCc1cc(-c2ncc(-c3cccc(O)c3O)o2)ccc1O] | −71.8 | <5 |
| LMH003 | 3-(2,3-dihydroxyphenyl)-1-(4- hydroxy-3- (hydroxymethyl)phenyl)propan- 1-one | 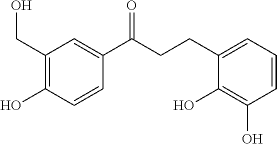 [SMILES: O=C(CCc1cccc(O)c1O)c1ccc(O)c(CO)c1] | −72.7 | <5 |
| LMH004 | 3-(2,3-dihydroxyphenyl)-1- (3,4-dihydroxyphenyl)propan- 1-one | 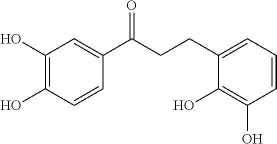 [SMILES: O=C(CCc1cccc(O)c1O)c1ccc(O)c(O)c1] | −51.0 | <5 |
| LMH005 | N-(2,3-dihydroxybenzyl)-4- hydroxy-3- (hydroxymethyl)benzamide | 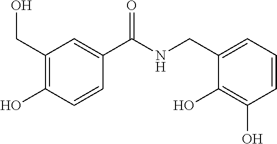 [SMILES: O=C(NCc1cccc(O)c1O)c1ccc(O)c(CO)c1] | −66.0 | <5 |
| LMH006 | N-(2,3-dihydroxybenzyl)-3,4- dihydroxybenzamide | 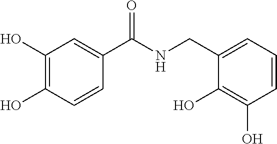 [SMILES: O=C(NCc1cccc(O)c1O)c1ccc(O)c(O)c1] | −65.4 | <5 |
| LMH007 | N-(2,3-dihydroxybenzyl)-3- hydroxy-4- (hydroxymethyl)benzamine | 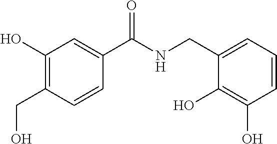 [SMILES: O=C(NCc1cccc(O)c1O)c1ccc(CO)c(O)c1] | −61.5 | <5 |
| LMH008 | 2,3-dihydroxybenzyl 4- hydroxy-3- (hydroxymethyl)benzimine | 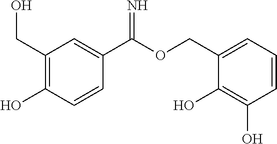 [SMILES: N=C(OCc1cccc(O)c1O)c1ccc(O)c(CO)c1] | −69.1 | <5 |
| LMH009 | 2,3-dihydroxybenzyl 4- hydroxy-3-(3- hydroxybenzyl)benzoate | 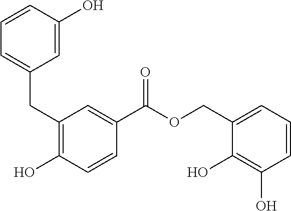 [SMILES: O=C(OCc1cccc(O)c1O)c1ccc(O)c(Cc2cccc(O)c2)c1] | −67.4 | <5 |
| LMH010 | 2,3-dihydroxybenzyl 3-benzyl- 4-hydroxybenzoate | 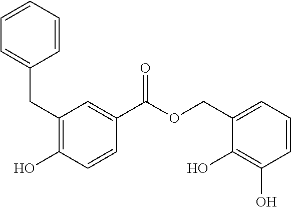 [SMILES: O=C(OCc1cccc(O)c1O)c1ccc(O)c(Cc2ccccc2)c1] | −64.8 | <5 |
| LMH011 | 2,3-dihydroxybenzyl 3- (hydroxymethyl)-4- (hydroxymethyl)benzoate | 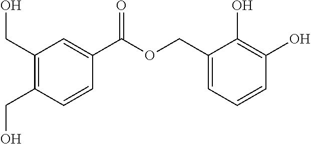 [SMILES: O=C(OCc1cccc(O)c1O)c1ccc(CO)c(CO)c1] | −65.1 | <5 |
| LMH012 | 2,3-dihydroxybenzyl 3,4- dihydroxybenzoate | 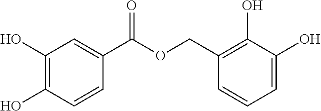 [SMILES: O=C(OCc1cccc(O)c1O)c1ccc(O)c(O)c1] | −59.5 | <5 |
| LMH013 | 2,3-dihydroxybenzyl 3- hydroxy-4-methylbenzoate | 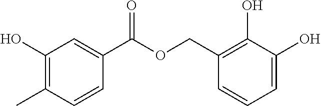 [SMILES: Cc1ccc(C(=O)OCc2cccc(O)c2O)cc1O] | −63.3 | <5 |
| LMH014 | 2,4-dihydroxybenzyl 3- hydroxy-4- (hydroxymethyl)benzoate | 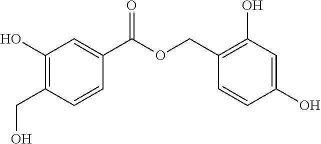 [SMILES: O=C(OCc1ccc(O)cc1O)c1ccc(CO)c(O)c1] | −62.6 | <5 |
| LMH015 | 2,3-dihydroxybenzyl 2- napthoate | 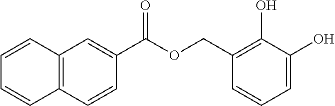 [SMILES: O=C(OCc1cccc(O)c1O)c1ccc2ccccc2c1] | −59.4 | <5 |
| LMH016 | 2,3-dihydroxybenzyl 6- hydroxy-7-(4-hydroxyphenyl)- 2-napthoate | 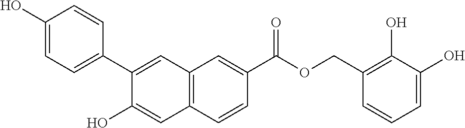 [SMILES: O=C(OCc1cccc(O)c1O)c1ccc2cc(O)c(-c3ccc(O)cc3)cc2c1] | −61.8 | <5 |
| LMH0107 | 2,3-dihydroxybenzyl 6- hydroxy-7-phenyl-2-napthoate | 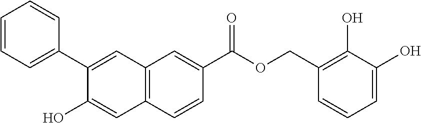 [SMILES: O=C(OCc1cccc(O)c1O)c1ccc2cc(O)c(-c3ccccc3)cc2c1] | −63.7 | <5 |
| LMH018 | 2,3-dihydroxybenzyl 6- hydroxy-7-methyl-2-napthoate | 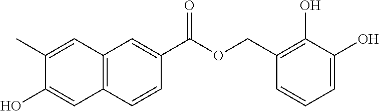 [SMILES: Cc1cc2cc(C(=O)OCc3cccc(O)c3O)ccc2cc1O] | −68.0 | <5 |
| LMH019 | 2,3-dihydroxybenzyl 6- hydroxy-7-(hydroxymethyl)-2- napthoate | 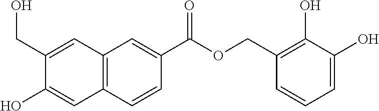 [SMILES: O=C(OCc1cccc(O)c1O)c1ccc2cc(O)c(CO)cc2c1] | −68.9 | <5 |
| LMH020 | 2,3-dihydroxybenzyl 6,7- dihydroxy-2-napthoate | 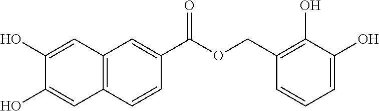 [SMILES: O=C(OCc1cccc(O)c1O)c1ccc2cc(O)c(O)cc2c1] | −67.2 | <5 |
| LMH021 | 2,3-dihydroxybenzyl 7- hydroxy-2-napthoate | 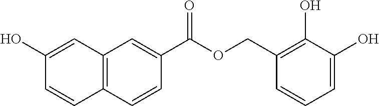 [SMILES: O=C(OCc1cccc(O)c1O)c1ccc2ccc(O)cc2c1] | −72.3 | <5 |
| LMH022 | 2,3-dihydroxybenzyl 6,8- dihydroxyisoquinoline-3- carboxylate | 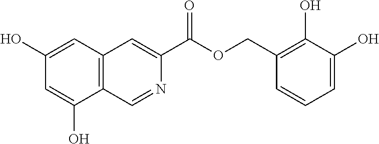 [SMILES: O=C(OCc1cccc(O)c1O)c1cc2cc(O)cc(O)c2cn1] | −72.3 | <5 |
| LMH023 | 2,3-dihydroxybenzyl 8- hydroxy-6- (hydroxymethyl)isoquinoline-3- carboxylate | 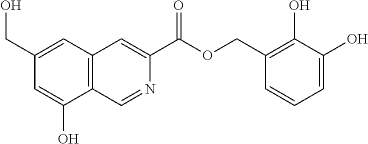 [SMILES: O=C(OCc1cccc(O)c1O)c1cc2cc(CO)cc(O)c2cn1] | −68.9 | <5 |
| LMH024 | (3,8-dihydroxynapthalen-2- yl)methyl 4-hydroxy-3- (hydroxymethyl)benzoate | 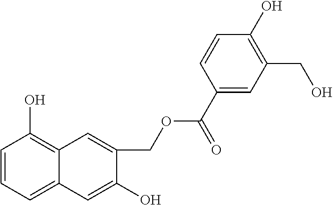 [SMILES: O=C(OCc1cc2c(O)cccc2cc1O)c1ccc(O)c(CO)c1] | −76.2 | <5 |
| LMH025 | 2,3-dihydroxybenzyl 1,5- dihydroxyisoquinoline-6- carboxylate | 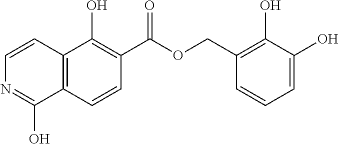 [SMILES: O=C(OCc1cccc(O)c1O)c1ccc2c(O)nccc2c1O] | −65.4 | <5 |
| LMH026 | (7,8-dihydroxynapthalen-2- yl)methyl 4-hydroxy-3- (hydroxymethyl)benzoate | 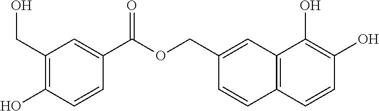 [SMILES: O=C(OCc1ccc2ccc(O)c(O)c2c1)c1ccc(O)c(CO)c1] | −80.8 | <5 |
| LMH027 | 4-(5-(2,3-dihydroxyphenyl)- 4,5dihydrooxazol-2- yl)naphthalene-2,8-diol | 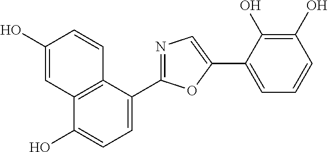 [SMILES: Oc1ccc2c(-c3ncc(-c4cccc(O)c4O)o3)ccc(O)c2c1] | −70.1 | <5 |
| LMH028 | 3-(2-(6-hydroxy-4- methylnapthalen-1-yl)oxazol-5- yl)benzene-1,2-diol | 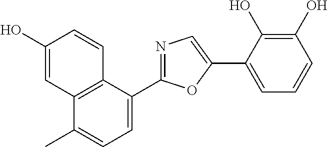 [SMILES: Cc1ccc(-c2ncc(-c3cccc(O)c3O)o2)c2ccc(O)cc12] | −67.8 | <5 |
| LMH029 | 3-(2-(6-hydroxy-4- (hydroxymethyl)napthalen-1- yl)oxazol-5-yl)benzene-1,2-diol | 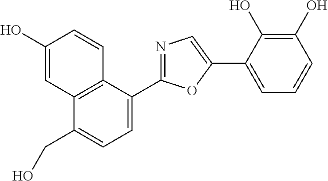 [SMILES: OCc1ccc(-c2ncc(-c3cccc(O)c3O)o2)c2ccc(O)cc12] | −59.1 | <5 |
| LMH030 | 3-(2-(napthalen-1-yl)oxazol-5- yl)benzene-1,2-diol | 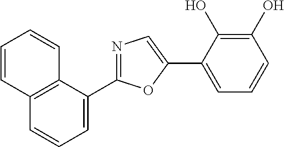 [SMILES: Oc1cccc(-c2cnc(-c3cccc4ccccc34)o2)c1O] | −51.6 | <5 |
| LMH031 | 3-(2-(6-hydroxy-7- methylnapthalen-1-yl)benzene- 1,2-diol | 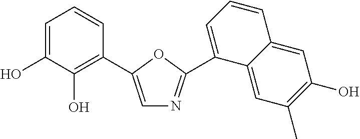 [SMILES: Cc1cc2c(-c3ncc(-c4cccc(O)c4O)o3)cccc2cc1O] | −71.1 | <5 |
| LMH032 | 3-(2-(6-hydroxy-7- (hydroxymethyl)napthalen-1- yl)oxazol-5-yl)benzene-1,2-diol | 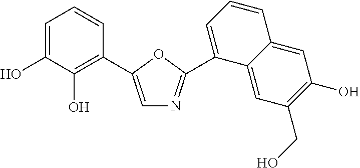 [SMILES: OCc1cc2c(-c3ncc(-c4cccc(O)c4O)o3)cccc2cc1O] | −68.1 | <5 |
| LMH033 | 5-(5-(2,3- dihydroxyphenyl)oxazol-2- yl)naphthalene-2,3-diol | 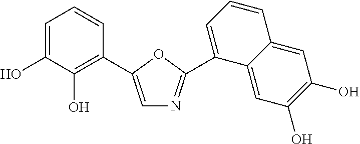 [SMILES: Oc1cc2cccc(-c3ncc(-c4cccc(O)c4O)o3)c2cc1O] | −74.9 | <5 |
| LMH034 | 3-(2-(7-hydroxy-6- methylnapthalen-1-yl)oxazol-5- yl)benzene-1,2-diol | 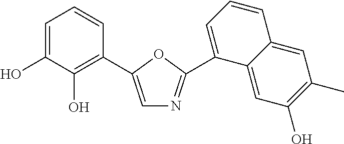 [SMILES: Cc1cc2cccc(-c3ncc(-c4cccc(O)c4O)o3)c2cc1O] | −66.5 | <5 |
| LMH035 | 3-(2-(napthalen-1-yl)oxazol-5- yl)benzene-1,2-diol | 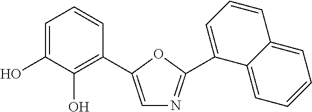 [SMILES: Oc1cccc(-c2cnc(-c3cccc4ccccc34)o2)c1O] | −52.6 | <5 |
| LMH036 | 3-(2-(4-hydroxy-3- (hydroxymethyl)phenyl)oxazol- 5-yl)benzene-1,2-diol | 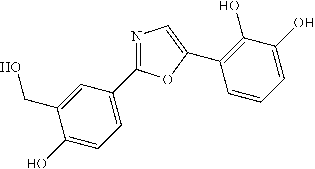 [SMILES: OCc1cc(-c2ncc(-c3cccc(O)c3O)o2)ccc1O] | −79.1 | <5 |
| LMH037 | 3-(2-(6-hydroxy-7- (hydroxymethyl)napthalen-2- yl)oxazol-5-yl)benzene-1,2-diol | 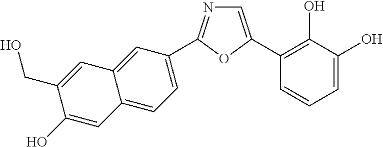 [SMILES: OCc1cc2cc(-c3ncc(-c4cccc(O)c4O)o3)ccc2cc1O] | −79.0 | <5 |
| LMH038 | 6-(5-(2,3- dihydroxyphenyl)oxazol-2- yl)napthene-2,3-diol | 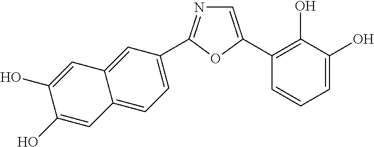 [SMILES: Oc1cc2ccc(-c3ncc(-c4cccc(O)c4O)o3)cc2cc1O] | −69.0 | <5 |
| LMH039 | 3-(2-(7-hydroxynapthalen-2- yl)oxazol-5-yl)benzene-1,2-diol | 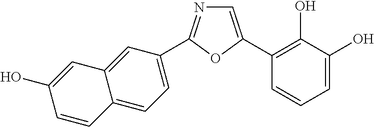 [SMILES: Oc1ccc2ccc(-c3ncc(-c4cccc(O)c4O)o3)cc2c1] | −58.0 | <5 |

Compounds which bind to proteins in a favourable conformation bring the complex to a lower energy state thus reducing the energy within the system. This reduction in energy is calculated and expressed as the binding energy (KJ/mol).

The compounds according to the invention may be tested for their activity in the inhibition or reduction of formation of ROS from oxygen in cells. The activity of the compounds is tested in the following cell cultures by different techniques according to the protocols detailed below.

### EXPLANATION OF ABBREVIATIONS

FCS (fetal calf serum), BSA (bovine serum albumin), DCF (2,7-dichlorodihydrofluorescein), EDTA, ROS (reactive oxygen species), SOD (superoxide dismutase), PMA (phorbol 12-myristate 13-acetate), O_{2.}⁻ (superoxide), U (unit), CO₂ (carbon dioxide), MLU (mean light unit), MTT (thiazolyl blue tetrazolium bromide), SD (standard deviation), EDTA (ethylenediaminetetra acetic acid), HBSS (Hank's buffered salt solution), H₂DCF-DA (2′,7′-dichlodihydrofluorescein diacetate), NADPH (nicotinamide adenine dinucleotide diphosphate), PBS (phosphate buffered saline), DMEM (Dulbecco's Modified Eagle Medium).

### 1. Cells and Cell Culture

### 1.1 Mouse Microvascular Endothelial Cells

The mouse lymph node microvascular endothelial cell line (SVEC4-10) was from the American Type Culture Collection (CRL-2167) and grown in DMEM, containing 10% (v/v) heat inactivated foetal calf serum, 100 U/mL penicillin and 100 mg/mL streptomycin, in a humidified atmosphere at 37° C. with 5% CO₂.

### 1.2 NIH-3T3 Fibroblasts

The NIH-3T3 fibroblast is a mouse embryonic fibroblast cell line. Cells were grown in DMEM containing, 10% (v/v) heat inactivated foetal calf serum, 100 U/mL penicillin and 100 mg/mL streptomycin, in a humidified atmosphere at 37° C. with 5% CO₂.

### 1.3 Primary Mouse Bone Marrow Hematopoietic Stem Cells

On the day of experiment, primary hematopoietic bone marrow cells were obtained from wild-type male C57BL/6 mice as described previously (11). Briefly, 12-16 week old mice were sacrificed by cervical dislocation and their hind legs dissected and carefully cleaned from adherent tissues. Next, the ends of each bone were removed to expose the marrow and this was collected by flushing the femurs and tibias using a 25 gauge needle loaded with Dulbecco's PBS. The bone marrow flushing was then loaded onto Histoplaque and spun at 400×g for 30 minutes to collect the hematopoietic bone marrow stem cells. Freshly isolated cells were used immediately for the experiments.

### 1.4 Human HL-60 Cells

HL-60 is a human promyelocytic leukaemia cell line. The HL60 cells were grown in RPMI 1640 medium; containing 10% (v/v) heat inactivated foetal calf serum, 100 U/mL penicillin and 100 mg/mL streptomycin, in a humidified atmosphere at 37° C. with 5% CO₂. HL60 cells were differentiated to the human neutrophils by culturing the cells in the culture medium in the presence of 1.25% v/v DMSO for 6 days before being used for the experiments.

### 1.5 IMR90 Cells

IMR90 is a human foetal lung fibroblast cell line. The IMR90 cells were grown in DMEM, containing 10% (v/v) heat inactivated foetal calf serum, 100 U/mL penicillin and 100 mg/mL streptomycin, in a humidified atmosphere at 37° C. with 5% CO₂.

### 1.6 HepG2 Cells

HepG2 is a human hepatocytic cell line (ATCC No. HB-8065) that was originally derived from liver tissue with a well differentiated hepatocellular carcinoma. HepG2 cells have been proven to be a suitable in vitro model system for the study of human hepatocytes, and the study of liver metabolism and toxicity. HepG2 cells were grown in DMEM, containing 10% (v/v) heat inactivated foetal calf serum, 100 U/mL penicillin and 100 mg/mL streptomycin, in a humidified atmosphere at 37° C. with 5% CO₂.

### 1.7 A549 Cells

A549 is a human lung adenocarcinoma epithelial cell line. A549 cells were grown in DMEM, containing 10% (v/v) heat inactivated foetal calf serum, 100 U/mL penicillin and 100 mg/mL streptomycin, in a humidified atmosphere at 37° C. with 5% CO₂.

### 2. Methods for the Detection of ROS Production

### 2.1 Lucigenin-chemiluminescence (12)

O₂⁻ production by different cell types was measured using lucigenin (5 μM)-chemiluminescence. A microplate luminometer (LumiStar, BMG) was used, which allows the examination of 96 samples at the same time with kinetic reading facilities. Cells were resuspended to a dilution of 10⁶/ml in modified HEPES buffer containing (mM) NaCl 140, KCl 5, MgCl₂ 0.8, CaCl₂ 1.8, Na₂HPO₄ 1, HEPES 25, Cells were distributed at 5×10⁴/well concentration onto the 96 well microplate. Dark-adapted lucigenin (5 μM) was added into the well through an auto-dispenser located in the dark chamber just before reading. The light emission was recorded over a 60 minute period and expressed as arbitrary MLU measured by the illuminometer. Each experiment was performed in triplicate and the mean of 3 blank readings was subtracted from each corrected value for each reading. In some experiments cells were also stimulated by the addition of PMA (0.1 μg/mL), TNFα (100 U/ml) or angiotensin II (200 nM) before the measurement of chemiluminescence. For the measurement of NADPH (Nox2 enzyme substrate)-dependent O_{2.}⁻ production by endothelial cells in suspension, NADPH (100 μM final) was added just before the addition of lucigenin.

O₂⁻ production was also studied using total cell homogenate (for the endothelial cells and the fibroblasts). Cultured endothelial cells or fibroblasts were washed twice with ice cold PBS, detached and resuspended (1×10⁷/ml) in modified HEPES buffer (described above). Cells were broken by homogenization, followed by sonication for 2×15 seconds and the unbroken cells were spun down by centrifugation at 200 g for 5 min. Soluble protein concentration was determined using a kit from Bio-Rad Laboratories Ltd. (Hertfordshire, U.K.). The protocol for measurement of O₂⁻ production by cell homogenates (100 μg protein/well) using lucigenin-luminescence was exactly as described above.

For assessment of the effect of LMH001 (final concentration 0-100 μM). on O_{2.}⁻ production by living cells or cell homogenates, LMH001 was added into the well and pre-incubated with the cells or cell homogenates at room temperature for 5 minutes before the addition of NADPH. For investigation into the effects of LMH001 on chronic ROS production, LMH001 was added directly into the culture medium for 1 hour prior to either TNFα (100 U/ml), angiotensin H (200 nM) or EGF (10 ng/ml) stimulation for 24 hours, followed by ROS detection as described above.

2.2 Cytochrome c Assay with or without SOD (13)

These experiments were performed using cell suspensions as described previously (Li et al., 1999). Briefly, intact cells were resuspended in DMEM without phenol red containing LMH001 (final concentration 0-10 μM), and incubated in 96-well-flat bottom culture plates (1×10⁵ cells/well) for 5 minutes at 37° C. in a humidified CO₂ incubator. Cytochrome c (250 μM final concentration) and NADPH (100 μM each) were added to the cells in the presence or absence of SOD (200 units/mL) and allowed to incubate for a further 30 min. Reduction of cytochrome c was measured by reading absorbance at 550 nm on a microplate reader. O₂⁻ production in nmol/1×10⁵ cells was calculated from the difference between absorbance of samples with or without SOD and the extinction coefficient for change of ferricytochrome c to ferrocytochrome c, i.e. 21.0 mmol.L⁻¹.cm⁻¹. In some experiments cells were also stimulated by the addition of PMA (0.1 μg/mL) or TNFα (100 U/ml) prior to the addition of Cytochrome c and NADPH.

### 3. Assessment of Cellular Cytotoxicity

### 3.1 Cell Viability Assessed by Trypan Blue Exclusion and Cell Number Counting

Cell viability and membrane integrity in the presence and absence of LMH001 were assessed using the trypan blue exclusion assay as described previously (15). Briefly, cells (1×10⁶) were seeded onto 10 cm² culture dishes and allowed to adhere for 4 hours in 10% (v/v) FCS medium. Subsequently, cells were incubated with LMH001(0-10 μM) for 24 hours in 5% (v/v) FCS medium. At the time of experiment, cells were harvested by 0.25% Trypsin/EDTA, pelleted, resuspended in serum free medium and counted using a haemocytometer in the presence of trypan blue (0.4%) solution. The number of viable cells was calculated as a percentage of total cells.

### 3.2 Thiazolyl Blue Tetrazolium Bromide (MIT) Assay for Cytotoxicity

The potential cytotoxicity of LMH001 on cultured endothelial cells (SVEC4-10) and liver cells (HepG2) was assessed using the MIT assay as described previously (16). Briefly, intact cells were resuspended in culture medium containing 2.5% (v/v) FCS, plated in 96-well-flat bottom culture plates (5×10⁴ cells/well) with LMH001 (final concentration 0-10 μM) and incubated at 37° C. in a humidified CO₂ incubator. Twenty-four hours after the addition of LMH001, MTT, a yellow tetrazolium reagent which is converted to formazan by living cells, was added directly into each well and incubated for a further 2 hours at 37° C. The medium was then aspirated and the insoluble blue formazan crystals were dissolved in DMSO. Absorbance was measured at 570 nm in an ELISA plate reader and the mean of 3 blank readings was subtracted from each corrected value.

### 3.3 Cellular Proliferation Assay (MTS)

The ex vivo proliferative capabilities of isolated BMSC or cultured cells were analyzed using the MTS proliferation assay. BMSC were suspended in Dulbecco's modified Eagle's medium supplemented with 5% fetal bovine serum (Sigma), 100 U/mL penicillin and 100 μg/mL streptomycin, and plated in 1% gelatin coated 96-well plates at a density of 1×10⁵ cells/well in triplicate. After 24 hours in culture medium containing LMH001 (0-10 μM), proliferation was assessed using the MTS (CellTiter 96 AQ; Promega, Madison, USA) assay according to the manufacturer's protocol. The absorbance at 490 nm was read using an ELISA plate reader (Perkin elmer), and the proliferation index calculated relative to the controls. The mean of 3 blank readings was subtracted from each corrected value.

### 4. Administration of LMH001 to C57BL/6J Mice

In vivo test of LMH001 on bone marrow haematopoietic stem cell proliferation was performed using wild-type male C57BL/63 mice (5 months of age). Mice were injected intraperitoneal with vehicle control (DMSO; 0.1%) or LMH001 (2 mg/kg/day) for 14 days. Soluble 0.5 molar solutions of LMH001 were first prepared in research grade DMSO followed by sub-dilutions with medical grade saline (0.9% NaCl). The proliferative capabilities of the isolated cells were analysed by the MTS assay as described above.

### 5. Statistical Analysis

Results were calculated as the mean±SD from triplicate measurements over 3 different cell culture experiments. The difference in the Of production by cells with or without inhibitors was analysed using Bonferonni t-test. IC₅₀ was calculated using Prism 5 (GraphPad software LTD).

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications are covered by the appended claims.

All references identified herein are incorporated herein by reference in their entirety. Full details of references (1) to (20) identified herein are provided below:
(1) Lambeth, J. D., Kawahara, T., and Diebold, B. (2007). Regulation of Nox and Duox exzymatic activity and expression. Free Radical Biology and Medicine 43, 319-331.
(2) Pagano, P. J. (2000). Vascular gp91(phox)—Beyond the endothelium. Circulation Research 87, 1-3.
(3) Drummond, G. R., Selemidis, S., Griendling, K. K., and Sobey, C. G. (2011). Combating oxidative stress in vascular disease; NADPH oxidases as therapeutic targets. Nature Reviews Drug Discovery 10, 453-471.
(4) Block, M. L. (2008). NADPH oxidase as a therapeutic target in Alzheimer's disease. BMC Neuroscience 9.
(5) Jay, D. Hitomi, H. Griendling, K K. (2005) Oxidative stress and diabetic cardiovascular complications. Free Radical Biology and Medicine. 40:2; 183-192.
(6) Asaba, K., Tojo, A., Onozato, M. L., Goto, A., Quinn, M. T., Fujita, T., and Wilcox, C. S. (2005). Effects NADPH oxidase inhibitor in diabetic nephropathy. Kidney International 67, 1890-1898.
(7) van der Vliet, A. (2008). NADPH oxidases in lung biology and pathology: Host defence enzymes, and more. Free Radical Biology and Medicine 44, 938-955.
(8) Gao, X. P., Standiford, T. J., Rahman, A., Newstead, M., Holland, S. M., Dinauer, M. C., Liu, Q. H., and Malik, A. B. (2002). Role of NADPH oxidase in the mechanism of lung neutrophil sequestration and microvessel injury induced by gram-negative sepsis: Studies in p47(phox−/−) and gp91(phox−/−) mice. Journal of Immunology 168, 3974-3982.
(9) Weintraub, N. L. (2002). Nox response to injury. Arteriosclerosis Thrombosis and Vascular Biology 22, 4-5.
(10) Krause, K. H. (2007). Aging: A revisited theory based on free radicals generated by NOX family NADPH oxidases. Experimental Gerontology 42, 256-262.
(11) Xu, S., De Becker, A., Van Camp, B., Vanderkerken, K., and Van Riet, I. (2010). An Improved Harvest and in vitro Expansion Protocol for Murine Bone Marrow-Derived Mesenchymal Stem Cells. Journal of Biomedicine and Biotechnology.
(12) Li, J. M., Mullen, A. M., Yun, S., Wientjes F., Brouns, G. Y., Thrasher, A. J., and Shah, A. M. (2002). Essential role of the NADPH oxidase subunit p47(phox) in endothelial cell superoxide production in response to phorbol ester and tumor necrosis factor-alpha, Circulation Research 90, 143-150.
(13) Li, J. M., and Shah, A. M. (2001). Differential NADPH-versus NADH-dependent superoxide production by phagocyte-type endothelial cell NADPH oxidase. Cardiovascular Research 52, 477-486.
(14) Xiao, D., Zeng, Y., Prakash, L., Badmaev, V., Majeed, M., and Singh, S. V. (2011). Reactive Oxygen Species-Dependent Apoptosis by Gugulipid Extract of Ayurvedlc Medicine Plant Commiphora mukul in Human Prostate Cancer Cells Is Regulated by c-Jun N-Terminal Kinase. Molecular Pharmacology 79, 499-507.
(15) Li, J.-M., Fan, L. M., George, V. T., and Brooks, G. (2007). Nox2 regulates endothelial cell cycle arrest and apoptosis via p21 (cip1) and p53. Free Radical Biology and Medicine 43, 976-986.
(16) Rastinejad, F., Conboy, M. J., Rando, T. A., and Blau, H. M. (1993). Tumor suppression by the TNA from the 3′ untranslated region of alpha-tropomyosin. Cell 75, 1107-1117.
(17) Besselievre, F., Mahuteau-Betzer, F., Grierson, D. S. & Piguel, S. (2008). Ligandess microwave-assisted Pd/Cu-catalyzed direct arylation of oxazoles. Journal of Organic Chemistry, 71
(18) Gisch, N., Balzarini, 3. & Meier, C. (2007). Enzymatically activated cycloSal-d4T-monophosphates: The third generation of cydoSal-pronucleotides. Journal of Medicinal Chemistry, 50.
(19) Yang, F., Xu, Z., Wang, Z., Yu, Z. & Wang, R. (2011). Copper-Catalyzed Oxidative Arylation of Heteroarenes under Mild Conditions Using Dioxygen as the Sole Oxidant. Chemistry-a European Journal, 17.
(20) Petersen, M. A., Deniz, E., Nielsen, M. B., Sortino, S, & Raymo, F. M. (2009). Photochromic Oxazines with Extended Conjugation. European Journal of Organic Chemistry.

1. A compound of the formula (I) 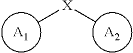
wherein
A₁ and A₂ are independently selected from: phenyl, pyridinyl, naphthyl and quinolinyl;
A₂ is substituted by at least two OH groups; and
A₁ and A₂ are optionally substituted by one or more groups selected from: H, OR¹⁴, NR¹⁵R¹⁶, R¹⁷ and halogen; wherein
R¹⁴, R¹⁵ and R¹⁶ are each independently selected from: H and alkyl;
R¹⁷ is alkyl, aryl or arylalkyl, where each group is optionally substituted by one or more groups selected from: OR¹⁴, Nee and halogen, where R¹⁴, R¹⁵ and R¹⁶ are as defined;
X is a 5 or 6-membered unsaturated, saturated or partially unsaturated carbocyclic or heterocyclic ring, wherein the heterocyclic ring has 1, 2, 3, or 4 heteroatoms selected from: O, N and S, or
X is a group of the formula below 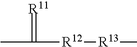
wherein
R¹¹ is O, S or NH; and
R¹² and R¹³ are independently selected from: O, S, NH and CH₂;
in any stereochemical form, or a mixture of any stereochemical forms in any ratios;
or a pharmaceutically acceptable salt, metabolite, prodrug, or a mixture thereof.

2. A compound according to claim 1 of the formula (II) below 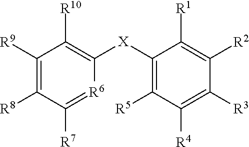
wherein
R¹ to R⁵ and R⁷ to R¹⁰ are each independently selected from: H, OR¹⁴, NR¹⁵R¹⁶, R¹⁷ and halogen; where
R¹⁴, R¹⁵ and R¹⁶ are each independently selected from: H and alkyl;
R¹⁷ is alkyl, aryl or arylalkyl, where each group is optionally substituted by one or more groups selected from: OR¹⁴, NR¹⁵R¹⁶ and halogen, where R¹⁴, R¹⁵ and R¹⁶ are as defined;
or
R² and R³, together with the respective carbon atoms to which they are attached, form a 6-membered unsaturated, or partially unsaturated carbocyclic or heterocyclic ring, wherein the heterocyclic ring has at least one N heteroatom, and wherein the carbocyclic or heterocyclic ring is optionally substituted by one or more groups selected from: OR¹⁴, NR¹⁵R¹⁶, R¹⁷ and halogen, where R¹⁴, R¹⁵, R¹⁶ and R¹⁷ are as defined;
or
R³ and R⁴, together with the respective carbon atoms to which they are attached, form a 6-membered unsaturated, or partially unsaturated carbocyclic or heterocyclic ring, wherein the heterocyclic ring has at least one N heteroatom, and wherein the carbocyclic or heterocyclic ring is optionally substituted by one or more of groups selected from: OR¹⁴, NR¹⁵, R¹⁶, R¹⁷ and halogen, where R¹⁴, R¹⁵, R¹⁶, and R¹⁷ are as defined;
or
R⁸ and R⁹, together with the respective carbon atoms to which they are attached, form a 6-membered unsaturated, or partially unsaturated carbocyclic or heterocyclic ring, wherein the heterocyclic ring has at least one N heteroatom, and wherein the carbocyclic or heterocyclic ring is optionally substituted by one or more groups selected from: OR¹⁴, NR¹⁵R¹⁶ R¹⁷ and halogen, where R¹⁴, R¹⁵, R¹⁶ and R¹⁷ are as defined;
or
R⁹ and R¹⁰, together with the respective carbon atoms to which they are attached, form a 6-membered unsaturated, or partially unsaturated carbocyclic or heterocyclic ring, wherein the heterocyclic ring has at least one N heteroatom, and wherein the carbocyclic or heterocyclic ring is optionally substituted by one or more groups selected from: OR¹⁴, NR¹⁵, R¹⁶, R¹⁷ and halogen, where R¹⁴, R¹⁵, R¹⁶, and R¹⁷ are as defined;
R⁶ is N or CH;
X is a 5 or 6-membered unsaturated, saturated or partially unsaturated carbocyclic or heterocyclic ring, wherein the heterocyclic ring has 1, 2, 3, or 4 heteroatoms selected from: O, N and S, or
X is a group of the formula below 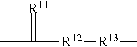
wherein
R¹¹ is O, S or NH; and
R¹² and R¹³ are independently selected from: O, S, NH and CH₂;
in any stereochemical form, or a mixture of any stereochemical forms in any ratios; or a pharmaceutically acceptable salt, metabolite, prodrug, or a mixture thereof.

3. A compound according to claim 2, wherein
R¹ to R⁵ and R⁷ to R¹⁰ are each independently selected from: H, OH, alkyl and benzyl, where the alkyl and benzyl groups are optionally independently substituted by 1, 2 or 3 groups selected from: OH and hydroxyalkyl;
or
R² and R³, together with the respective carbon atoms to which they are attached, form a 6-membered unsaturated carbocyclic ring, optionally substituted by one or more groups selected from: OH, alkyl and hydroxyalkyl;
or
R³ and R⁴, together with the respective carbon atoms to which they are attached, form a 6-membered unsaturated carbocyclic ring, optionally substituted by one or more groups selected from: OH, alkyl and hydroxyalkyl;
or
R⁸ and R⁹, together with the respective carbon atoms to which they are attached, form a 6-membered unsaturated or partially unsaturated carbocyclic or heterocyclic ring, wherein the carbocyclic or heterocyclic ring is optionally substituted by one or more groups selected from: OH, alkyl, hydroxyalkyl, benzyl, benzyl substituted by 1 or 2 OH, phenyl, phenyl substituted by 1 or 2 OH;
or
R⁹ and R¹⁰, together with the respective carbon atoms to which they are attached, form a 6-membered unsaturated carbocyclic ring, optionally substituted by one or more groups selected from: OH, alkyl and hydroxyalkyl;
R⁶ is N or CH;
X is a 5-membered unsaturated or partially unsaturated heterocyclic ring having 1, 2 or 3 heteroatoms selected from: O, N and S, or
X is a group of the formula below 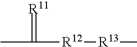
wherein
R¹¹ is O or NH;
R¹² is O, NH or CH₂; and
R¹³ is CH₂;
in any stereochemical form, or a mixture of any stereochemical forms in any ratios; or a pharmaceutically acceptable salt, metabolite, prodrug, or a mixture thereof.

4. A compound according to any one of claims 1 to 3 of the formula (III) below 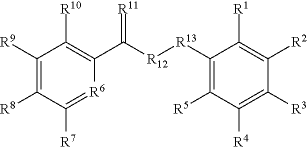
wherein R¹ to R⁵ and R⁷, R⁸ and R¹⁰ are independently selected from: H, OH, alkyl and hydroxyalkyl;
R⁹ is selected from: H, OH, alkyl and benzyl, where the alkyl and benzyl groups are optionally independently substituted by 1 or 2 OH;
or
R² and R³, together with the respective carbon atoms to which they are attached, form a 6-membered unsaturated carbocyclic ring, optionally substituted by one or more groups selected from: OH, alkyl and hydroxyalkyl;
or
R³ and R⁴, together with the respective carbon atoms to which they are attached, form a 6-membered unsaturated carbocyclic ring, optionally substituted by one or more of the groups: OH, alkyl and hydroxyalkyl;
or
R⁸ and R⁹, together with the respective carbon atoms to which they are attached, form a 6-membered unsaturated or partially unsaturated carbocyclic or heterocyclic ring, optionally substituted by one or more of the groups: OH, alkyl and hydroxyalkyl, benzyl, benzyl substituted by 1 or 2 OH, phenyl, phenyl substituted by 1 or 2 OH;
R⁶ is N or CH;
R¹¹ is O or NH;
R¹² is O, NH or CH₂; and
R¹³ is CH₂;
in any stereochemical form, or a mixture of any stereochemical forms in any ratios; or a pharmaceutically acceptable salt, metabolite, prodrug, or a mixture thereof.

5. A compound according to any one of claims 1 to 3 of the formula (IV) below 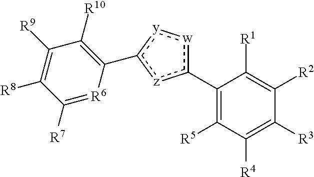
wherein
R¹ to R⁵ and R⁷ to R¹⁰ are independently selected from: H, OH, alkyl and hydroxyalkyl;
or
R⁸ and R⁹, together with the respective carbon atoms to which they are attached, form a 6-membered unsaturated carbocyclic ring, optionally substituted by one or more groups selected from: OH, alkyl and hydroxyalkyl;
or
R⁹ and R¹⁰, together with the respective carbon atoms to which they are attached, form a 6-membered unsaturated carbocyclic ring, optionally substituted by one or more groups selected from: OH, alkyl and hydroxyalkyl;
R⁶ is N or CH;
the atoms z, y and w are independently selected from: C, O, N and S;
the dashed fine represents an optional bond;
in any stereochemical form, or a mixture of any stereochemical forms in any ratios; or a pharmaceutically acceptable salt, metabolite, prodrug, or a mixture thereof.

6. A compound according to any one of the preceding claims 2 to 5, wherein
R¹ and R² are OH and R³, R⁴ and R⁵ are H; or
R⁴ and R⁵ are OH and R¹, R² and R³ are H.

7. A compound according to any one of the preceding claims 2 to 6, wherein R⁸ is methyl, hydroxymethyl or OH; R⁹ is methyl, hydroxymethyl or OH; and R⁷ and R¹⁰ are H.

8. A compound which is selected from:
2,3-dihydroxyphenyl)methyl 4-hydroxy-3-(hydroxymethyl)benzoate;
3-(2,3-dihydroxyphenyl)-1-(4-hydroxy-3-(hydroxymethyl)phenyl)propan-1-one;
3-(2,3-dihydroxyphenyl)-1-(3,4-dihydroxyphenyl)propan-1-one;
N-(2,3-dihydroxybenzyl)-4-hydroxy-3-(dihydroxymethyl)benzamide;
N-(2,3-dihydroxybenzyl)-3,4-dihydroxybenzamide;
N-(2,3-dihydroxybenzyl)-3-hydroxy-4-(hydroxmethyl)benzamine;
2,3-dihydroxybenzyl 4-hydroxy-3-(hydroxymethyl)benzimine;
2,3-dihydroxybenzyl 4-hydroxy-3-(3-hydroxybenzyl)benzoate;
2,3-dihydroxybenzyl 3-benzyl-4-hydroxybenzoate;
2,3-dihydroxybenzyl 3-(hydroxymethyl)-4-(hydroxymethyl)benzoate;
2,3-dihydroxybenzyl 3,4-dihydroxybenzoate;
2,3-dihydroxybenzyl 3-hydroxy-4-methylbenzoate;
2,4-dihydroxybenzyl 3-hydroxy-4-(hydroxymethyl)benzoate;
2,3-dihydroxybenzyl 2-napthoate;
2,3-dihydroxybenzyl 6-hydroxy-7-(4-hydroxyphenyl)-2-napthoate;
2,3-dihydroxybenzyl 6-hydroxy-7-phenyl-2-napthoate;
2,3-dihydroxybenzyl 6-hydroxy-7-methyl-2-napthoate;
2,3-dihydroxybenzyl 6-hydroxy-7-(hydroxymethyl)-2-napthoate;
2,3-dihydroxybenzyl 6,7-dihydroxy-2-napthoate;
2,3-dihydroxybenzyl 7-hydroxy-2-napthoate;
2,3-dihydroxybenzyl 6,8-dihydroxyisoquinoline-3-carboxylate; and
2,3-dihydroxybenzyl 8-hydroxy-6-(hydroxymethyl)isoquinoline-3-carboxylate;
3,8-dihydroxynapthalene-2-yl)methyl 4-hydroxy-3-(hydroxymethyl)benzoate;
2,3-dihydroxybenzyl 1,5-dihydroxyisoquinoline-6-carboxylate; and
7,8-dihydroxynapthalene-2-yl)methyl 4-hydroxy-3-(hydroxymethyl)benzoate;

9. A compound which is selected from:
3-(2-(4-hydroxy-3-(hydroxymethyl)phenyl)-1H-oxazol-5-yl)benzene-1,2-diol;
4-(5-(2,3-dihydroxyphenyl)-4,5dihydrooxazol-2-yl)naphthalene-2,8-diol;
3-(2-(6-hydroxy-4-methylnapthalen-1-yl)oxazol-5-yl)benzene-1,2-diol;
3-(2-(6-hydroxy-4-(hydroxymethyl)napthalen-1-yl)oxazol-5-yl)benzene-1,2-diol;
3-(2-(napthalen-1-yl)oxazol-5-yl)benzene-1,2-diol;
3-(2-(6-hydroxy-7-methylnapthalen-1-yl)benzene-1,2-diol;
3-(2-(6-hydroxy-7-(hydroxymethyl)napthalen-1-yl)oxazol-5-yl)benzene-1,2-diol;
5-(5-(2,3-dihydroxyphenyl)oxazol-2-yl)naphthalene-2,3-diol;
3-(2-(7-hydroxy-6-methylnapthalen-1-yl)oxazol-5-yl)benzene-1,2-diol;
3-(2-(4-hydroxy-3-(hydroxymethyl)phenyl)oxazol-5-yl)benzene-1,2-diol;
3-(2-(napthalen-1-yl)oxazol-5-yl)benzene-1,2-diol;
3-(2-(6-hydroxy-7-(hydroxymethyl)napthalen-2-yl)oxazol-5-yl)benzene-1,2-diol;
6-(5-(2,3-dihydroxyphenyl)oxazol-2-yl)napthalene-2,3-diol; and
3-(2-(7-hydroxynapthalen-2-yl)oxazol-5-yl)benzene-1,2-diol.

10. A pharmaceutical composition comprising a compound according to any one of claims 1 to 9, and a pharmaceutically acceptable carrier, diluent or excipient.

11. A compound according to any one of claims 1 to 9 for use as a medicament.

12. A compound according to any one of claims 1 to 9 for use in the treatment of a disease or condition selected from: cardiovascular diseases, respiratory diseases, inflammatory diseases, cancers, ageing and age related disorders, kidney diseases, neurodegenerative diseases, diabetes and conditions associated with diabetes.

13. A method for treating a subject suffering from a disease or condition selected from: cardiovascular diseases, respiratory diseases, inflammatory diseases, cancers, ageing and age related disorders, kidney diseases, neurodegenerative diseases, diabetes and conditions associated with diabetes, the method comprising administering to the subject in need thereof a compound as claimed in any one of claims 1 to 9.

14. A process for the preparation of a compound as claimed in any one of claims 1 to 4 and 6 to 9, comprising reacting a compound of the formula (V) 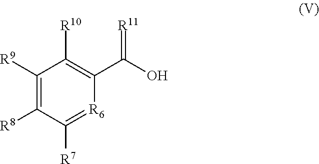
wherein R⁶ to R¹¹ are as defined, optionally wherein any one or more of the groups R⁷ to R¹⁰ are protected, with a compound of the formula (VI) 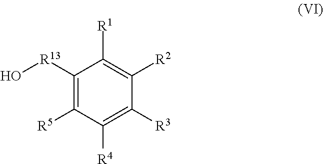
wherein R¹ to R⁵ and R¹³ are as defined, optionally wherein any one or more of the groups R¹ to R⁵ are protected, to form a compound of the formula (III), 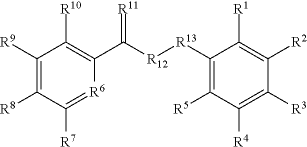
after removal of any protecting groups, wherein R¹² is O, and R¹ to R¹¹ and R¹³ are as defined, and, optionally, converting the compound into a pharmaceutically acceptable salt.

15. A process for the preparation of a compound of the formula (X) below 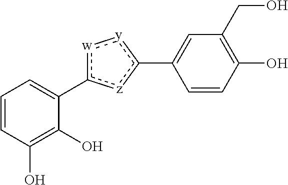
wherein w, y and z are as defined and the dashed line represents an optional bond, comprising reacting a compound of the formula (XI) 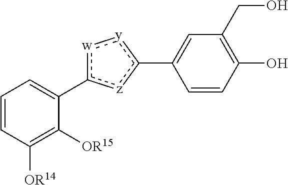
wherein R¹⁴ and R¹⁵ are protective groups, with dry 10% Pd/carbon, in an organic solvent, optionally in the presence of triethylamine, to form the compound of the formula (X), and, optionally, converting the resultant compound into a pharmaceutically acceptable salt.
